(19) United States
(12) Patent Application Publication
(10) Pub. No.: US 2013/0183761 A1
(43) Pub. Date: Jul. 18, 2013

(54) Methods for Incorporating Unnatural Amino Acids in Eukaryotic Cells

(71) Applicants: Jason Chin, Cambridge (GB); Alexandre Deiters, Raleigh, NC (US); Rajendra Uprety, Raleigh, NC (US); Susan M. Hancock, Cambridge (GB); Sebastian Greiss, Cambridge (GB)

(72) Inventors: Jason Chin, Cambridge (GB); Alexandre Deiters, Raleigh, NC (US); Rajendra Uprety, Raleigh, NC (US); Susan M. Hancock, Cambridge (GB); Sebastian Greiss, Cambridge (GB)

(73) Assignees: North Carolina State University, Raleigh, NC (US); Medical Research Council, Swindon (GB)

(21) Appl. No.: 13/825,720

(22) PCT Filed: Sep. 23, 2011
(86) PCT No.: PCT/GB2011/001392

§ 371 Date: Mar. 22, 2013

(30) Foreign Application Priority Data
Sep. 24, 2010 (GB) 1016143.8
Jul. 7, 2011 (GB) 1111661.3

(51) Int. Cl. C12N 15/52

(52) U.S. Cl. CPC C12N 15/52
USPC 435/471; 536/23.2; 435/254.21; 435/325; 435/348; 435/354; 435/366

(57) ABSTRACT

The invention relates to a nucleic acid comprising a nucleotide sequence encoding a tRNA orthogonal to a eukaryotic cell, said nucleotide sequence operably linked to a promoter capable of directing transcription by eukaryotic RNA polymerase III. The invention also relates to methods for incorporating unnatural amino acids in eukaryotic cells using same.

### INTRODUCTION

The pyrrolysl-tRNA synthetase/tRNA (PylRS/tRNA_{CUA}^{Pyl}) pairs from M. barkeri (Mb) and M. mazei (Mm) are orthogonal in E. coli.¹ These pairs have been evolved to direct the site-specific incorporation of a range of unnatural amino acids, including amino acids that are post-translationally modified, amino acids containing bio-orthogonal chemical handles, and amino acids protected with light and acid sensitive groups into proteins in E. coli in response to the amber codon.¹⁻⁶ In contrast to other aminoacyl-tRNA synthetase/tRNA pairs for the incorporation of unnatural amino acids, which are orthogonal in either eukaryotic or prokaryotic hosts, the PylRS/tRNA pairs are orthogonal in both E. coli and mammalian cells.^{2,6,7} Several unnatural amino acids have been site-specifically incorporated into proteins in mammalian cells by evolving the synthetase/tRNA pair in E. coli and subsequently transferring it to mammalian cells. This approach has the advantage of bypassing the requirement to evolve the amino acid specificity of the synthetase directly in a eukaryotic host.^{8,10} Another advantage of said approach is the versatility of the variants of pyrrolysl-tRNA synthetase that can be used in the orthogonal pair to site specifically incorporate a variety of unnatural amino acids.

Many biological processes are more effectively addressed in the yeast Saccharomyces cerevisiae (S. cerevisiae) than in mammalian cells. Yeast has a rapid doubling time, bar-coded libraries of gene knockouts exist, protein interaction and transcriptome data is most complete, tap-tagged strains are readily available and powerful genetic approaches can be simply implemented. However the requirement to evolve the current orthogonal pairs directly in yeast has limited the scope of unnatural amino acids that have been incorporated in yeast.

Preliminary work by Yokoyama and coworkers introduced a PylRS/tRNA_{CUA}^{Pyl}, pair into yeast and reported very weak phenotypes consistent with poor incorporation of N_{ε}-tert-butyl-oxycarbonyl-L-lysine,⁷ but a properly characterized system for incorporating amino acids using the PylRS/tRNA_{CUA}^{Pyl} pair has not been reported.

### SUMMARY OF THE INVENTION

Here we report the creation and characterization of functional and orthogonal tRNA synthetase-tRNA pairs (such as pyrrolysyl-tRNA synthetase/tRNAPyl pairs) in a eukaryote such as yeast. We demonstrate the incorporation of several useful unnatural amino acids using variants of this pair created in E. coli.

In another aspect the invention provides an orthogonal PylRS/tRNA^{Pyl} pair that is functional in a eukaryote such as yeast for site-specifically incorporating unnatural amino acids into proteins.

In another aspect, the invention relates to a nucleic acid comprising a nucleotide sequence encoding a tRNA orthogonal to a eukaryotic cell, said nucleotide sequence operably linked to a promoter capable of directing transcription by eukaryotic RNA polymerase III.

At least three aminoacyl-tRNA synthetase/tRNA_{CUA} pairs (EcTyrRS/tRNA_{CUA}, EcLeuRS/tRNA_{CUA} and PylRS/tRNA_{CUA} from methanosarcina species) are orthogonal in eukaryotic cells and can be used to incorporate unnatural amino acids. In mammalian, worm (nematode) and fly systems, suitably PylRS/PylT are used. In worm (nematode) and fly systems suitably the M. mazei versions are used. In worm (nematode) and fly systems suitably the synthetase is wild type or the PCKRS mutant, most suitably wild type. In fly systems suitably the PCKRS mutant may be used.

Suitably said orthogonal tRNA is tRNA^{Pyl}.

As a practical matter, when said orthogonal tRNA is tRNA^{Pyl}, it should be noted that the PylT gene used may however lack the three 3′ bases (CCA), since in eukaryotes these are added post-transcriptionally. Suitably the wild type PylRS is used for multicellular eukaryote systems.

Suitably said eukaryotic cell is a yeast cell and the tRNA^{Pyl} comprises sequence at positions 3 and 70 which do not form a 3-70 base pair.

In another aspect, the invention relates to a nucleic acid comprising a nucleotide sequence encoding tRNA^{Pyl} operably linked to a promoter capable of directing transcription by yeast RNA polymerase III, wherein the tRNA^{Pyl} comprises sequence at positions 3 and 70 which do not form a 3-70 base pair.

Suitably the tRNA^{Pyl} comprises adenosine at position 3.

Suitably the yeast is Saccharomyces cerevisiae.

RNA POL III Promoter

Suitably any promoter capable of directing RNA Pol III transcription in eukaryotic cells may be used.

Various options for RNA Pol III promoters are described throughout the specification, including intragenic and extragenic (internal and external) promoters.

Suitably the promoter comprises A and B box consensus sequences.

Suitably said promoter is, or is derived from, the eukaryotic U6 promoter.

An exemplary U6 promoter is described in Das, G., Henning, D., & Reddy, R. (1987). Structure, organization, and transcription of Drosophila U6 small nuclear RNA genes. The Journal of biological chemistry, 262(3), 1187-1193.

An exemplary U6 promter for use in human and/or mouse systems is described in Gautier, A., Nguyen, D. P., Lusic, H., An, W., Deiters, A., & Chin, J. W. (2010). Genetically encoded photocontrol of protein localization in mammalian cells. Journal of the American Chemical Society, 132(12), 4086-4088

C. elegans Pol III promoters which may find application in the invention (also the primer sequences to amplify them from genomic worm DNA) are presented in the following table:

| | | Direc- |
|---|---|---|
| Promoter | Primer Sequence | tion |
| rpr-1 | CGATTTCGGCTAAAAAATAGCG | F |
| (Y48G10A.7) | | |
| rpr-1 | ATACAACTTGACGCGCGCCGCGTCG | R |
| (Y48G10A.7) | | |
| CeN124 | GCGGTCTAGACCGGGTACCG | F |
| (F55A11.10) | | |
| CeN124 | ATGATAGGTGTCGAACTAGCGGG | R |
| (F55A11.10) | | |
| CeN68 (Y59E1B) | GACAAGGTAAGTCTACAGGC | F |
| CeN68 (Y59E1B) | ATTCAAATGATGAATTGATAAATAGTT | R |
| | TATGG | |
| CeN88 (Y37E3) | GGAGGTCGCGGTAACGCCGG | F |
| CeN88 (Y37E3) | ATGGTGTGTTTTGAAACAAGTCAAACA | R |
| | AGAA | |
| CeN102 | CCATATGCAAACTGATTCTCG | F |
| (Y46G5A.40) | | |
| CeN102 | ATTTAGCTAATTTTTGATGCAAAAATCG | R |
| (Y46G5A.40) | | |
| CeN103 | CGCCCGCCACTGAGGCTCCG | F |
| (F26E4.14) | | |
| CeN103 | ATTACTTTGGAAATTATTGGGAAAATTG | R |
| (F26E4.14) | | |
| CeN31 | GACTAGGATATGCTAAACCTTGCC | F |
| (Y45F10B.19) | | |
| CeN31 | ATGATTGAAATACTGACAGAATGGGGG | R |
| (Y45F10B.19) | | |

An exemplary RNA Pol III promoter may be used as follows: Drosophila melanogaster U6-2 snRNA gene, complete sequence.

| ACCESSION | | M24606 |
|---|---|---|
| VERSION | | M24606.1 GI:1708929 |
| KEYWORDS | U6 small nuclear RNA. | |
| FEATURES | | Location/Qualifiers |
| | promoter | 1..401 |
| | | /note=Dm U6-2 Promoter |
| | tRNA | 402..470 |
| | | /label=M. mazei tRNA Pyl |
| | | /note=ApEinfo_fwdcolor=cyan |
| | | /note=ApEinfo_revcolor=green |
| | 3′UTR | 476..562 |

/note=Dm U6-2 3′ region

| | |
|---|---|
| BASE | 174 A 113 C 82 G 192 T |
| COUNT | |
| 0 OTHER | |
| ORIGIN | ? |
| 1 | TGTTCGACTT GCAGCCTGAA ATACGGCACG AGTAGGAAAA |

GCCGAGTCAA ATGCCGAATG

| | |
|---|---|
| 61 | CAGAGTCTCA TTACAGCACA ATCAACTCAA GAAAAACTCG |

ACACTTTTTT ACCATTTGCA

| | |
|---|---|
| 121 | CTTAAATCCT TTTTTATTCG TTATGTATAC TTTTTTTGGT |

CCCTAACCAA AACAAAACCA

| | |
|---|---|
| 181 | AACTCTCTTA GTCGTGCCTC TATATTTAAA ACTATCAATT |

TATTATAGTC AATAAATCGA

| | |
|---|---|
| 241 | ACTGTGTTTT CAACAAACGA ACAATGGACA CTTTGATTCT |

AAAGGAAATT TTGAAAATCT

| | |
|---|---|
| 301 | TAAGCAGAGG GTTCTTAAGA CCATTTGCCA ATTCTTATAA |

TTCTCAACTG TCTCTTTCCT

| | |
|---|---|
| 361 | GATGTTGATC ATTTATATAG GTATGTTTTC CTCAATACTT |

CGGAAACCTG ATCATGTAGA

| | |
|---|---|
| 421 | TCGAATGGAC TCTAAATCCG TTCAGCCGGG TTAGATTCCC |

GGGGTTTCCG TTTTTTTGCT

| | |
|---|---|
| 481 | AACCTGTGAT TGCTCCTACT CAAATACAAA AACATCAAAT |

TTTCTGTCAA TAAAGCATAT

| | |
|---|---|
| 541 | TTATTTATAT TTATTTTACA GG |

Another exemplary RNA Pol III promoter may be used as follows:

| | |
|---|---|
| promoter | 1..452 |
| | /note=322 Ce N74-1 |
| gene | <453..454 |
| | /locus_tag=C03B1.2 |
| | /db_xref=WormBase:WBGene00015373 |
| | /label=gene(2) |
| | /note=ApEinfo_label=gene |
| | /note=ApEinfo_fwdcolor=#ff0906 |
| | /note=ApEinfo_revcolor=green |
| tRNA | 455..523 |
| | /label=M. mazei tRNA Pyl |
| | /note=ApEinfo_fwdcolor=cyan |
| | /note=ApEinfo_revcolor=green |
| source | <524..1569 |
| | /3′ region of C. elegans sup-7 C0381. |
| | t1) |
| gene | <524..1569 |
| | /locus_tag=C03B1.2 |
| | /db_xref=WormBase:WBGene00015373 |
| | /label=gene |
| | /note=ApEinfo_fwdcolor=#ff0906 |
| | /note=ApEinfo_revcolor=green [Split] |
| BASE | 532 A 261 C 266 G 509 T |
| COUNT | |
| 0 OTHER | |
| ORIGIN | ? |
| 1 | CAGTTTAACA AGGGCTTCAA ACATCGTTCA AAAGAGTTTG |

GCACGACATA TAATTCGAGA

| | |
|---|---|
| 61 | TGCAATGGGA ATGGTGTTAT CTCCAGAAAG TTTTGAATTT |

TTTAAAAGCT GTTTTAAGCT

| | |
|---|---|
| 121 | TTGTCTTCGC ATATCTTGTA AACATTTGTA TTTTTAAATA |

CATTTTCGTA TTTAAGAGAC

| | |
|---|---|
| 181 | AGTTTTTTTG GATGTTTGAC AAACCATGTT GCCTTCAGTT |

TTAGAAATGT TCAATGTAAC

| | |
|---|---|
| 241 | CATACAAAAT CATGTGGGAT CATTTTCTTG CTTCAGGTAA |

AACTTTAAAA CGTTCCGAAC

| | |
|---|---|
| 301 | GCTATTTGGA GTTGCAAATG GTCTATGTCC AAATAAATTT |

GTGGAAATTG CGAAATACTT

| | |
|---|---|
| 361 | CATAACAGAA CCAGAGGTTG CGCAATTGCA CGAAAAATGT |

CTGCCGCCTG ACTCTCCTTC

| | |
|---|---|
| 421 | GGTATATAAA CAGCCTACAA TCACCTACCC GTATGGAAAC |

CTGATCATGT AGATCGAATG

| | |
|---|---|
| 481 | GACTCTAAAT CCGTTCAGCC GGGTTAGATT CCCGGGGTTT |

CCGAATTTTT TGTTTTTTAA

| | |
|---|---|
| 541 | GTAGTAATAT AATACAATTT AATTCAAAAT TACACGCAAA |

AATTTAATAA AGTAGTCCAA

| | |
|---|---|
| 601 | AATGCTAATC GTGTGAAAAA ATGCCTTCTG GTAATGTATT |

TCTCACACGA AAATTTGTTG

| | |
|---|---|
| 661 | AAATTGGTTT TTGACACAAG CGAATGTTGC AATTATTTTC |

TTGATTTTTC TACAAATTTG

| | |
|---|---|
| 721 | AAAATCAAAT TTCACGACTG CAAGTATAAC ATTAAAAAAC |

AATATACCCA AGCCCACAAA

| | |
|---|---|
| 781 | AAAACGTATT CTGAAAAATT GGTACAATTG AGAAATAAAA |

TTTGAGAATC CCTTATCTTT

| | |
|---|---|
| 841 | AGAAGTTGAT CTCTTGATAT TGATACAACT CATGCTAGAC |

AGTGTCTACT CTGCTCAAAA

| | |
|---|---|
| 901 | AGTCAATTAG TAATTTGATC CGCTAGCAAT CTAACGCATA |

GGATGCACCC GCAAACTGAA

| | |
|---|---|
| 961 | CACCTATATC CAGACAATTG TTGCGCAATA ATGTCTAAGT |

CTCCTGTATT TGTGTGTATT

| | |
|---|---|
| 1021 | TTACGAAACA TGTAACGGAT AGGAATTTTG CTGACCTCAC |

ACGTATGGAT GATGTGCAAC

| | |
|---|---|
| 1081 | AACTTGCGAT TAAGGATAGG ATATGTGTCA ATTAGAGCAA |

ATGGATTATA CTAATGAAAA

| | |
|---|---|
| 1141 | CAATTGAAGA GTTTTTTACC TTTACGGAAA TCTTTATAAT |

TAGTTCAACT TGAACAACGG

| | |
|---|---|
| 1201 | GAATTTAATT TTTTTTTTAA CATGGGATAA ATGAAATAAT |

TAAGATGAAC AAATTTCAGC

| | |
|---|---|
| 1261 | CAACTCCGCC GAGACAGTCG TATTGCATTG TCAGATGCAA |

TTTTCCAGCC GGAAAAGAAG

| | |
|---|---|
| 1321 | ACAAACACGA TGGATCATTT ATTTACCGCT TCTACAGCTC |

CGGAATACTC GGAGAAGGAA

| | |
|---|---|
| 1381 | TTGCGACAGA TAGAAGATTT TCAGTCATTT TTTGACAATT |

GGGATGATGA TTACTATACG

| | |
|---|---|
| 1441 | GAGCAAGGTA CTTTTAGAAT TTCTGAAATA TTTTTACAAA |

AATTACTATT TTTAATTCTA

| | |
|---|---|
| 1501 | GAAAAATATC CAGTTATATA TGACCTCATA ACAATTCAAA |

TTTCAGATTG TCTAGACCTG

| | |
|---|---|
| 1561 | ATGCAACAA |

A most preferred nucleic acid of the invention comprises a U6 promoter capable of directing RNA Pol III transcription in mammalian cells such as mouse or human cells operably linked to tRNA^{Pyl}, more suitably tRNA^{Pyl}_{cua}.

Suitably the promoter comprises the yeast sequence encoding tRNA^{Arg}_{UCU}.

Suitably the tRNA^{Pyl} is tRNA^{Pyl}_{CUA}.

Suitably the sequence encoding tRNA^{Pyl}_{CUA} comprises the M. mazei tRNA^{Pyl}_{CUA} sequence.

An exemplary sequence of M. mazei tRNA_{CUA} is as follows: the 3′ CCA that is added post-transcriptionally in eukaryotes (and therefore may be omitted as is the case in the gene in the expression constructs in the examples section) is indicated in BOLD:

GGAAACCUGAUCAUGUAGAUCGAAUGGACUCUAAAUCCGUUCAGCCGG
GUUAGAUUCCCGGGGUUUCCGCCA

Suitably the sequence encoding tRNA^{Pyl}_{CUA} comprises the M. barkeri tRNA^{Pyl}_{CUA} sequence having a G3A substitution.

In another aspect, the invention relates to an expression system comprising a nucleic acid as described above; said system further comprising a nucleotide sequence encoding a PylRS capable of aminoacylating the tRNA^{Pyl}.

Suitably the PylRS comprises M. barkeri PylRS or AcKRS or TfaKRS or PcKRS.

Suitably the synthetase comprises PylRS such as M. mazei PylRS. An exemplary sequence of M. mazei PylRS is shown. The sequence in BOLD is a FLAG tag which is optionally included to be able to easily detect the protein on a western blot.

MDYKDDDDKDKKPLNTLISATGLWMSRTGTIHKIKHHEVSRSKIYIEMA
CGDHLVVNNSRSSRTARALRHHKYRKTCKRCRVSDEDLNKFLTKANEDQ
TSVKVKVVSAPTRTKKAMPKSVARAPKPLENTEAAQAQPSGSKFSPAIP
VSTQESVSVPASVSTSISSISTGATASALVKGNTNPITSMSAPVQASAP
ALTKSQTDRLEVLLNPKDEISLNSGKPFRELESELLSRRKKDLQQIYAE
ERENYLGKLEREITRFFVDRGFLEIKSPILIPLEYIERMGIDNDTELSK
QIFRVDKNFCLRPMLAPNLYNYLRKLDRALPDPIKIFEIGPCYRKESDG
KEHLEEFTMLNFCQMGSGCTRENLESIITDFLNHLGIDFKIVGDSCMVY
GDTLDVMHGDLELSSAVVGPIPLDREWGIDKPWIGAGFGLERLLKVKHD
FKNIKRAARSESYYNGISTNL

In another aspect, the invention relates to a eukaryote such as a yeast cell comprising a nucleic acid as described above or an expression system as described above.

Suitably the yeast cell is S. cerevisiae.

In another aspect, the invention relates to use of a nucleic acid as described above or an expression system as described above to incorporate an unnatural amino acid into a protein in a eukaryote such as a yeast cell.

In another aspect, the invention relates to a method for incorporating an unnatural amino acid into a protein in a eukaryote cell such as a yeast cell comprising the following steps:
i) introducing an expression system as described above into said cell;
ii) introducing a nucleic acid encoding the protein into said cell, said nucleic acid comprising an orthogonal codon recognised by the orthogonal tRNA (such as tRNA^{Pyl}) of the expression system at the position for incorporation of the unnatural amino acid; and
iii) incubating the eukaryote cell such as yeast cell in the presence of unnatural amino acid to be incorporated.

In another aspect, the invention relates to a method as described above, wherein the unnatural amino acid to be incorporated is an alkyne-containing amino acid or a post-translationally modified amino acid or an amino acid containing bio-orthogonal chemical handles or a photo-caged amino acid or a photo-crosslinking amino acid.

PylRS is pyrrolysyl-tRNA synthetase. This may typically be an Archaea PylRS such as a Methanosarcina PylRS such as a M. barkeri or M. mazei PylRS, or a PylRS derived from same. PylRS derived from a M. barkeri or M. mazei PylRS may include acetyl-lysyl-tRNA synthetase (AcKRS) or Trifluoro-acetyl-lysyl-tRNA synthetase (TfaKRS) or photocaged Lysyl-tRNA synthetase (PcKRS) as discussed below.

Suitably, the PylRS is derived from M. barkeri.

Suitably the nucleotide sequence encoding the PylRS is codon optimised for a eukaryote such as a yeast such as S. cerevisioe.

Suitably the orthogonal tRNA of the invention is tRNA^{Pyl}.

A preferred example of a tRNA^{Pyl} of the invention is the tRNA^{Pyl} of M. mazei.

Suitably the tRNA^{Pyl} is tRNA_{CUA}^{Pyl}. This incorporates unnatural amino acids by amber suppression i.e. by recognition of the amber codon.

Suitably the tRNA^{Pyl} is operably linked to a promoter for transcription by eukaryotic RNA polymerase III, such as yeast RNA polymerase III. Suitably the promoter comprises the sequence encoding for yeast tRNA_{UCU}^{Arg} (alternatively described in the art as tDNA).

The eukaryote (or eukaryotic cell) may be any eukaryote (or from any eukaryote) such as yeast, flies (e.g. Drosophila such as Drosophila melanogaster), nematodes (e.g. C. elegans), mice (e.g. Mus musculus), humans or other eukaryote.

The RNA Pol III promoter may be from any source such as any eukaryote provided that it retains the ability to direct transcription of the tRNA in a eukaryote (or eukaryotic cell).

The invention also provides a eukaryote cell such as a yeast cell comprising the PylRS/tRNA^{Pyl} pair of the invention. Suitably, the yeast cell is S. cerevisiae, more preferably S. cerevisiae MaV203.

The introduction of the PylRS to a eukaryote such as yeast cell may be done according to any method known in the art, suitably by transforming a nucleotide to sequence encoding the PylRS into the eukaryote cell.

The invention also relates to the use of an orthogonal tRNA synthetase/tRNA pair such as a PylRS/tRNA^{Pyl} pair to incorporate unnatural amino acids into proteins in a eukaryote such as a yeast cell. This could be alternatively described as a method for incorporating unnatural amino acids into proteins in a eukaryote such as a yeast cell comprising the following steps: transforming the eukaryote such as yeast cell with a nucleotide sequence or sequences encoding an orthogonal tRNA synthetase/tRNA pair such as PylRS and tRNA^{Pyl} as described above and then placing the eukaryote such as yeast cell in medium containing the unnatural amino acid to be incorporated.

Given the variants available for PylRS, as detailed above and discussed below, the PylRS/tRNA^{Pyl} pair is an especially versatile use or method of incorporating unnatural amino acids in yeast because it can be used to incorporate an alkyne-containing amino acid or a post-translationally modified amino acid or an amino acid containing bio-orthogonal chemical handles or a photo-caged amino acid or a photo-cross linking amino acid.

Preferably, said incorporation is done through amber suppression and thus with a tRNA_{CUA}^{Pyl}.

### DETAILED DESCRIPTION OF THE INVENTION

Genetic code expansion has been limited to the incorporation of unnatural amino acids in cultured cells and unicellular organisms. Here we report genetic code expansion in eukaryotes. In addition we demonstrate this in multicellular eukaryotic animals, such as the nematode C. elegans

Suitably the pyrrolysyl-tRNA synthetase/tRNA^{Pyl} pair function as an orthogonal aminoacyl-tRNA synthetase/tRNA pair to incorporate unnatural amino acids into proteins in a eukaryote such as yeast with site specificity i.e. in response to a codon recognised by the tRNA^{Pyl}.

Within the context of the present invention, yeast means a eukaryotic microorganism classified in the Kingdom Fungi, with about 1,500 species described. Most reproduce asexually by budding, although a few reproduce by binary fission. Yeasts generally are unicellular, although some species may become multicellular through the formation of a string of connected budding cells known as pseudohyphae, or false hyphae. Exemplary yeasts that can be used in the disclosed methods and kits include but are not limited to Saccharomyces cerevisiae, Candida albicans, Schizosaccharomyces pombe, and Saccharomycetales. Most suitably the yeast is Saccharomyces cerevisiae.

The tRNA^{Pyl} suitably comprises sequence at positions 3 and 70 which do not form a 3-70 base pair; more suitably the tRNA^{Pyl} comprises adenosine at position 3 to achieve this. The absence of this base pair has the advantage of avoiding interference with yeast alanyl-tRNA synthetase. Thus this feature provides orthogonality. Suitably the tRNA” derives from M. mozei as this is an example of tRNA^{Pyl} that works with pyrrolysyl-tRNA synthetase (or its variants).

The expression “derived from” means that the nucleic acid or protein is based on or corresponds to the nucleic acid or protein recognised in the art as wild-type for the sequence of interest. The actual origin of the nucleic acid or protein is immaterial to the scope of the invention. There are many alternatives in the art to produce or isolate sequences of nucleic acid or protein, and the person skilled in the art is capable of choosing the most advantageous or suitable for his needs. Suitably derived from means at least 70% sequence identity to, more suitably at least 80% sequence identity to, more suitably at least 90% sequence identity to, more suitably at least 95% sequence identity to, more suitably at least 97% sequence identity to, more suitably at least 98% sequence identity to, more suitably at least 99% sequence identity to the sequence from which it is derived.

Within the context of the present invention pyrrolylsyl-tRNA synthetase or its variants (PylRS) is a group of aminoacyl-tRNA synthetases that possess a common protein structure but which may have been adapted (mutated) to carry different unnatural amino acids. The common protein structure is wild-type pyrrolylsyl-tRNA synthetase derived from M. barkeri (MbPylRS). Suitable PylRS species include AcKRS (a variant of MbPylRS that has been evolved to use 2³), TfaKRS (a variant of MbPylRS that can use 3, see text), PcKRS (a variant of MbPylRS that has been evolved to use 4². Thus the invention advantageously allows the incorporation of a wide variety of unnatural amino acids into proteins made in a eukaryote such as yeast with site specificity.

The use of a pyrrolylsyl-tRNA synthetase or its variants (PylRS) derived from M. barkeri and the use of amber suppression system permits the variation pyrrolylsyl-tRNA synthetases as discussed above.

This pairing of aminoacyl-tRNA synthetase and tRNA is advantageously used in a eukaryote such as yeast cells. Preferably said yeast cells are S. cerivisiae. These are the most studied yeast cells and most used in current molecular biology and biotechnology experimentation—fields where the present invention finds applications. The invention relates to the provision of eukaryotic cells such as yeast cells with an orthogonal pairing that comprises tRNA^{Pyl}. A preferred method of providing said eukaryotic cell with the orthogonal pairing is by providing nucleotides, preferably deoxyribonucleotides, that encode the synthetase protein and the tRNA^{Pyl}. Within the context of the present invention, “encode” refers to any process whereby the information in the sequence of a polymeric macromolecule is used to direct the production of a second molecule or sequence. This process can include transcription or translation, the operation of which in eukaryotic cells such as yeast cells is well known.

Both the synthetase protein and the tRNA^{Pyl} are preferably derived from prokaryotes or archaea. Methods of inducing the expression of a heterologous protein in eukaryotic cells are well known and therefore can be easily done for the synthetase. tRNA^{Pyl} is derived from prokaryotes and is transcribed in eukaryotic cells according to the present invention. A tRNA is suitably transcribed in eukaryotic cells by RNA Polymerase III. Thus another aspect of the present invention is the provision of a nucleotide that allows for the transcription of tRNA^{Pyl} in eukaryotic cells. This is suitably done according to the present invention by operably linking the nucleotide sequence encoding tRNA^{Pyl} to a promoter for RNA Polymerase

Suitably the nucleotides encoding the orthogonal pairing according to the invention, especially tRNA^{Pyl}, are deoxyribonucleotides.

Within the context of the present invention, promoter means a region of DNA that generally is located upstream (towards the 5′ region of a gene) that is needed for transcription. The promoter of the present invention for the tRNA is suitably for eukaryotic RNA Polymerase III. When the transcript comprises a leader RNA sequence, suitably the leader is subsequently cleaved post-transcriptionally from the primary transcript to yield the mature RNA product. The leader sequence may comprise one in which A- and B-boxes are internal to the primary transcript, but are external to the mature RNA product. As shown herein, internal promoters can be exploited to express E. coli tRNAs in eukaryotes such as yeast.

However, in other aspects of the invention the RNA Pol III promoter is suitably external to the transcribed RNA sequence. Incorporation of internal RNA polymerase III promoters into the transcribed section of a tRNA gene can affect the tertiary structure of the resulting tRNA. This can be by insertion and/or by substitution (mutation) but clearly in either case the resulting tRNA sequence has been altered when the RNA Pol III promoter is incorporated internally to the tRNA sequence. For this reason, it is advantageous to avoid altering the DNA sequence encoding the tRNA sequence to incorporate internal promoter(s). Suitably the RNA Pol III promoter is external to the tRNA coding sequence. Suitably the RNA Pol III promoter operably linked to the tRNA coding sequence is an extragenic promoter. Suitably the RNA Pol III promoter is 5′ to the tRNA coding sequence. The use of RNA Pol III promoters which are external to the tRNA sequence offers the advantage that the sequence of the tRNA is not affected by being operably linked to the RNA Pol III promoter.

Suitably said RNA Pol III promoters may include the SNR52 promoter, the RPR1 promoter or the SNR6 promoters. More suitably the promoter comprises tDNA_{UCU}^{Arg} tDNA_{UC}^{Arg} is a deoxyribonucleotide sequence which is part of a dicistronic gene which derives from yeast and codes for two mature tRNAs in yeast: tRNA_{UCU}^{Arg}-tRNA_{GUC}^{Asp}. tDNA_{UCU}^{Arg} is easily separable from the tDNA_{GUC}^{Asp} for example as described herein.

The promoter is operably linked to the deoxyribonucleotide encoding the tRNA^{Pyl}via any method known in the art. Preferably, it is attached by a 10-15 nucleotide bridge, for example as disclosed in FIG. 3 and/or Example 4. Preferably, it is attached to the 5′ end of the nucleotide sequence encoding the orthogonal tRNA such as tRNA^{Pyl}. Suitably the RNA Pol III promoter comprises a U6 promoter, i.e. an RNA Pol III promoter associated with the U6 small RNA in eukaryotic cells. The exact sequence of the U6 promoter varies between eukaryotes such as yeast, flies (e.g. Drosophila such as Drosophila melanogaster), nematodes (e.g. C. elegans), mice (e.g. Mus musculus), humans or other eukaryotes. In principle any of the U6 promoter sequences may be used in the invention. Suitably the RNA Pol III promoter is, or is derived from, a U6 promoter. More suitably the RNA Pol III promoter is, or is derived from, a human or mouse U6 promoter, most suitably a human U6 promoter. If a sequence derived from, but not 100% identical to, a wild type U6 promoter is used then it may be easily tested in the system(s) described herein to confirm that it retains the necessary promoter activity. This is well within the ability of the skilled worker.

As already mentioned above, the PylRS/tRNA^{Pyl} pairing in a yeast cell is another aspect of the invention. The PylRS/tRNA^{Pyl} are preferably created in E. coli. Any method known in the art can be used to introduce them to yeast cells, either together or separately. It is preferable if both are introduced into the yeast cell as deoxyribonucleotide sequences encoding for the PylRS protein and tRNA^{Pyl}, and that then these are transcribed by the yeast cell. Most suitably the sequences may be present on the same nucleic acid such as a plasmid.

The yeast cell could also be a cell that is part of a stable yeast cell line with the orthogonal pair according to the invention or nucleotides encoding for said pairing present in the yeast cell line.

The methods described herein rely upon the introduction of foreign or exogenous nucleic acids into yeast. Methods for yeast transformation with exogenous deoxyribonucleic acid, and particularly for rendering cells competent to take up exogenous nucleic acid, are well known in the art. The preferred method is the lithium acetate method.

The present invention allows incorporation of unnatural amino acids site specifically into proteins in yeast. The advantages of said orthogonal system include that it allows such incorporation to be done without otherwise disrupting the cell and thus to study the effects of the incorporation in vivo in yeast cells. Some examples of said uses are:
amino acid 1 may be used for bio-orthogonal 3+2 cycloadditions in yeast proteins;²⁴
amino acid 2 may be used for producing acetylated proteins directly in yeast and synthetically controlling processes normally regulated by acetylation in yeast;
amino acid 3 is a very poor substrate for sirtuins but not FIDACs²⁵ and should allow one to install irreversible acetylation at sites directly regulated by sirtuins in vivo in yeast cells. Thus it should allow one to probe the deacetylases that act on a given site in a protein;
amino acid 4 is a photocaged lysine with demonstrated utility for controlling protein function in eukaryotic cells² and one can easily anticipate that genetically-encoded photocontrol of proteins in yeast will be a powerful approach for gaining a temporal and spatial understanding of cellular processes;
amino acid 5 is a photocrosslinking amino acid with demonstrated utility mapping protein interactions in E. coli²³ which can find wide utility in mapping protein-protein interactions in yeast.

Given the growing list of amino acids that can be incorporated using MbPylRS and its variants,¹⁻⁶ it is clear that the introduction of a wide range of chemical functional groups into proteins in yeast via the invention is of wide industrial application.
Amino acid 6 is particularly suitable for multicellular eukaryotic applications.
Amino acids 7 and 8 are shown in FIG. 17. These have the advantage of providing alkyne groups for modification. These are particularly suitable for multicellular eukaryotic applications

Further Applications

The cells are suitably in vitro cells. Suitably the methods of the invention are in vitro methods. Suitably any test animals used are used in laboratory setting. Suitably the methods of the invention are not methods of treatment or surgery of the human or animal body.

In some embodiments, the cells may be comprised by a whole organism. In some embodiments the methods of making polypeptide incorporating unnatural amino acids may take place in the cells within an organism. Suitably such an organism is a multicellular eukaryote.

The invention also relates to systems and/or kits comprising the elements for incorporation of unnatural amino acids into polypeptides in eukaryotes according to the present invention. In particular such a system or kit may have three components:
(i) a nucleic acid comprising a nucleotide sequence encoding a tRNA orthogonal to a eukaryotic cell, said nucleotide sequence operably linked to a promoter capable of directing transcription by eukaryotic RNA polymerase III; and
(ii) a nucleic acid comprising a nucleotide sequence encoding the polypeptide of interest, said nucleic acid comprising an orthogonal codon recognised by the orthogonal tRNA (such as tRNA^{Pyl} cua) at the position for incorporation of the unnatural amino acid; and
(iii) a quantity of the unnatural amino acid to be incorporated.

In one embodiment (i) and (iii) may be provided on the same nucleic acid.

The coding sequence of (ii) is suitably operably linked to its own promoter. This promoter is suitably a promoter for RNA pol II, i.e. the conventional RNA polymerase use to express polypeptide coding sequences in eukaryotes. The coding sequence of (ii) may further be linked to a stabilising 3′ untranslated region (3′UTR) to stabilise the RNA in a eukaryotic cell. Thus in one embodiment the nucleic acid of (ii) comprises in the order 5′ to 3′; promoter, suitably RNA pol II promoter; coding sequence for polypeptide of interest comprising orthogonal codon at position for incorporation of unnatural amino acid; stabilising sequence such as stabilising 3′UTR sequence.

The invention also relates to new selectable marker constructs in nematodes such as C. elegans. In another aspect, the invention relates to a method for producing a nematode comprising a recombinant nucleic acid, said method comprising:
(i) preparing said recombinant nucleic acid such that it comprises a gene capable of expressing hygromycin B phosphotransferase;
(ii) introducing said nucleic acid to one or more cell(s) of said nematode
(iii) incubating said nematode in a medium comprising hygromycin B.

A challenge presented by multicellular eukaryotes is getting the unnatural amino acid into their cells to be available for incorporation. One method is to include the unnatural amino acid in the medium in which the multicellular eukaryotes live or grow. Another approach is to include the unnatural amino acid in their food. In this embodiment suitably the food comprises bacteria and suitably the unnatural amino acid is contacted with the bacteria; in this manner the unnatural amino acid is introduced to the multicellular eukaryote via the bacteria taking it up and being consumed by the multicellular eukaryote.

### BRIEF DESCRIPTION OF THE DRAWINGS

FIG. 1. Genetically-encoded incorporation of new unnatural amino acids in yeast. A. Unnatural amino acids used in this study. B. Amber suppression by foreign tRNAs in yeast. (a) The tRNA gene is transcribed by RNA polymerase III using A and B box promoter sequences internal to the structural gene; (b) Processing of tRNA precursor involving cleavage of 5′ and 3′ ends and addition of 3 ‘-CCA; (c) Export to cytoplasm for aminoacylation by aminoacyl-tRNA synthetases with an unnatural amino acid; (d) Ribosome mediated incorporation of the unnatural amino acid in response to an amber codon on the mRNA; (e) Production of a full length protein containing an unnatural amino acid at the genetically defined site.

FIG. 2. Creating a functional tRNA_{CUA}^{Pyl} in yeast. A. The consensus A and B box sequences and the A and B box sequences of MbtDNA_{CUA}^{Pyl}. B. The MbtDNA expression constructs created and examined in this work. Constructs 6a-d were created using the 5’ and 3′ flanks from distinct tRNAs as described in the text. C. Northern blots for MbtDNA expression from various constructs. D. Phenotyping constructs for amber suppression in MaV203:pGADGAL4(2TAG) cells where 3-AT is 3-aminotriazole and 1 was used at 2 mM. Cells contained MbPylRS and the appropriate MbtDNA_{CUA}^{Pyl} expression construct.

FIG. 3. MmtDNA_{CUA}^{Pyl} is orthogonal in yeast but MbtDNA directs the incorporation of alanine and is not orthogonal in yeast. A. Constructs used to compare orthogonality of tRNA_{CUA}^{Pyl} in yeast. B. Analysis of amber suppression by expression of hSOD33TAG-His₆ and detection by anti-His₆ western blot. Yeast cells containing the hSOD expression plasmid, MbPylRS and the dicistronic SctDN_{UCU}^{Arg}-tDNA_{CUA}^{Pyl} construct were grown in the presence or absence of 1 (5 mM). C. ESI-MS shows that alanine is incorporated into hSOD33TAG in cells producing amber suppressor MbtDNA_{CUA}^{Pyl} from construct 7 (Found 16553±1.5 Da, expected 16553 Da), confirming that MbtDNA_{CUA}^{Pyl} is a substrate for yeast alanyl-tRNA synthetases.

FIG. 4. Characterization of unnatural amino acid incorporation in yeast with the orthogonal MbPylRS/MmtDNA_{CUA}^{Pyl} pair. A. Amber suppression efficiency of hSOD33TAG-His₆ in yeast in the presence or absence of 1 (5 mM), 2 (10 mM), 3 (10 mM), 4 (2 mM), or 5 (1.3 mM) by anti-His₆ western blot. Yeast cells containing the hSOD expression construct were transformed with the dicistronic SctDNA_{UCU}^{Arg}-MmtDNA_{CUA}^{Pyl} construct for expressing the orthogonal MmtDNA_{CUA}^{Pyl} in yeast and the appropriate aminoacyl-tRNA synthetase (aaRS). PylRS (wild-type MbPylRS), AcKRS (a variant of MbPylRS that has been evolved to use 2³), TfaKRS (a variant of MbPylRS that can use 3, see text), PcKRS (a variant of MbPylRS that has been evolved to use 4²). B. Coomassie SDS-PAGE analysis of purified hSOD from expressions in the presence and absence of 1, 2 or 3. C-H. Full protein MS (C-E) and Glu-C MS/MS (F-H) confirms the incorporation of unnatural amino acids 1 (C/F found 16691±1.5 Da, expected 16691 Da), 2 (D/G found 16651±1.5 Da, expected 16651) and 3 (E/H found 16705±1.5 Da, expected 16705) at the genetically encoded site. hSOD is co-purified as a heterodimer with yeast SOD (minor additional peak in spectra at 15722 Da, identity confirmed by tryptic MS/MS). For full gels and western blots and larger versions of MS and MS/MS data see FIG. 6.

FIG. 5. A longer exposure of the northern blot shown in FIG. 2C is showing transcription of MbtDNA_{CUA}^{Pyl}. Constructs 3 and 4, possessing the B box mutations, show a small amount of MbtDNA_{CUA}^{Pyl} transcription. Constructs 6a also shows some tRNA trasncription.

FIG. 6. Whole western blot and SDS-PAGE gel as shown in FIGS. 4A and B and mass spectra shown in FIG. 4C-H. Characterization of unnatural amino acid incorporation in yeast with the orthogonal MbPylRS/MmtDNA_{CUA}^{Pyl} pair A. Amber suppression efficiency of hSOD33TAG-His₆ in yeast in the presence or absence of 1 (5 mM), 2 (10 mM), 3 (10 mM), 4 (2 mM), or 5 (1.3 mM) by anti-His₆ western blot. Yeast cells containing the hSOD expression construct were transformed with the dicistronic SctDNA_{UCU}^{Arg}-MmtDNA_{CUA}^{Pyl} construct for expressing the orthogonal MmtDNA_{CUA}^{Pyl} in yeast and the appropriate aminoacyl-tRNA synthetase (aaRS). PylRS (wild-type MbPylRS), AcKRS (a variant of MbPylRS that has been evolved to use 2³), TfaKRS (a variant of MbPylRS that can use 3, see text), PcKRS (a variant of MbPylRS that has been evolved to use 4²). Coomassie SDS-PAGE analysis of purified hSOD from expressions in the presence and absence of 1, 2 or 3. C-H. Full protein MS (C-E) and Glu-C MS/MS (F-H) confirms the incorporation of unnatural amino acids 1 (C/F found 16691±1.5 Da, expected 16691 Da), 2 (DIG found 16651±1.5 Da, expected 16651) and 3 (E/H found 16705±1.5 Da, expected 16705) at the genetically encoded site. hSOD is co-purified as a heterodimer with yeast SOD (minor additional peak in spectra at 15722 Da, identity confirmed by tryptic MS/MS).

FIG. 7.Expanding the genetic code of C. elegans. (A). Schematic of genetic code expansion in C. elegans. Amino acid (red star) is taken-up by the animal. In cells in the animal (grey oval) the synthetase (orange rectangle) aminoacylates the tRNA (red trident) with the unnatural amino acid. The tRNA is decoded on the ribosome (grey) in response to the amber stop codon, and the amino acid is incorporated into a polypeptide chain composed of otherwise natural amino acids (black circles). (B) DNA constructs used for genetic code expansion in C. elegans.

FIG. 8. Each of the components required for genetic code expansion in C. elegans can each be expressed in the animal (A) The effect of nonsense mediated decay (NMD) on GFP expression levels from worms containing the reporter construct (Prps-0::mGFP-TAG-mCherry-HA-NLS). +NMD shows the GFP fluorescence of a representative wild type animal. −NMD, shows the GFP fluorescence of a transgenic worm created by crossing the reporter construct into the smg-2(e2008) mutant background. (B) FLAG-MmPylRS (left panel) and MmtRNA_{CUA} (right panel) are expressed from animals containing Prps-0::FLAG-MmPylRS and PCeN74-1::MmPylT respectively. FLAG-MmPylRS was detected by western blot in worm to lysates using an anti-FLAG antibody. MmtRNA_{CUA} was detected by northern blot from total RNA isolated from worms. All experiments used a mixed stage population.

FIG. 9. The orthogonal MmPylRS/MmtRNA_{CUA} pair incorporates (6) in response to the amber codon in C. elegans (A) Representative fluorescence images of worms containing Ex1[Prps-0::mGFP-TAG-mcherry-HA-NLS, Prps-0::FLAG-MmPylRS; PCeN74-1::MmPylT; Prps-0::hpt] in the absence (top panels) and in the presence (bottom panels) of (6), see also Supplementary Movies 1-4). (B) Lanes 1-6: western blot of raw lysates from mixed populations of worms grown in the absence or presence of (6). Lanes 7 and 8: western blot of GFP::mCherry fusion protein affinity purified using an antibody against mcherry from worms grown in the absence or presence of (6). GFP::mCherry was detected using an antibody against the C-terminal HA tag. Western blots using anti-GFP were performed as loading controls (lanes 1-6) and input controls (lanes 7 and 8). Two independent lines were assayed. More protein was loaded in the no added amino acid lanes (lanes 1 and 4).

FIG. 10. (A) fluorescence imaging of worms carryin Ex1[Prps-0::mGFP-TAG-mcherry-HA-NLS, Prps-0::FLAG-MmPylRS, PCeN74-1::MmPylT, Prps-0::hpt)] transgenic array in a wild type and in smg-2(e2008) mutants that lack nonsense mediated decay. (B) complete scans of the blot shown in FIG. 2B Northern blot using a probe specifically recognizing MmtRNA_{CUA}; Lanes: M. 2-log biotinylated DNA ladder (New England Biolabs), 1. Positive control (MmtDNA_{CUA}), 2. Total RNA isolated from wild type, non-transgenic worms, 3. Total RNA isolated from worms carrying the Ex1 array. 40 μg of total RNA were loaded in lanes 2 and 3, 0.1 ng of MmtDNA_{CUA} was loaded in lane 1. The arrow indicates MmtDNA_{CUA} and MmtDNA_{CUA}. (C) Loading control for the northern blot. Equal amounts of the samples used for northern blotting were subjected to gel electrophoresis under identical conditions to those used for the northern blot and subsequently stained with SYBR Green II (Invitrogen). Lanes: 1 Total RNA isolated from wild type, non-transgenic worms, 2. Total RNA isolated from worms carrying the Ex1[Prps-0::mGFP-TAG-mcherry-HA-NLS, Prps-0::FLAG-MmPylRS, PCeN74-1::MmPylT, Prps-0::hpt)] array. (D) complete scan of western blot shown in FIG. 2B. Lanes: 1. Wild type, non-transgenic worms, 2. Worms carrying the Ex1[Prps-0::mGFP-TAG-mcherry-HA-NLS, Prps-0::FLAG-MmPylRS, PCeN74-1::MmPylT, Prps-0::hpt)] extra-chromosomal array. The arrow indicates PylRS.

FIG. 11.

(A) Western blot of lysates from mixed populations of worms grown in the absence or presence of (1). Antibodies against GFP were used to detect full length GFP-mCherry protein (upper panel) and GFP truncated at the amber stop codon (lower panel). (B) affinity purification of GFP-mCherry used in FIG. 3 (lanes 7 and 8). Lanes: 1-4 lysates of worms grown without (1), 1: raw lysate, 2: lysate after centrifugation to remove insoluble debris, 3: supernatant after binding to RFP-binder beads, 4:protein eluted from beads. Lanes 5-6 were loaded with samples corresponding to lanes 1-4, but with lysates of worms grown in the presence of (1). Anti-HA antibodies against the C-terminal HA tag were used to detect the fusion protein.

Supplementary Movies 1-4.

Animals carrying the Ex1[Prps-0::mGFP-TAG-mcherry-HA-NLS, Prps-0::FLAG-MmPylRS, PCeN74-1::MmPylT, Prps-0::hpt)] extra-chromosomal array were grown for 48 h in the presence (movies 1 & 2) or absence (movies 3 & 4) of (1). Movies were acquired using filter sets for mCherry and GFP. Channels were switched during movie acquisition, active filter sets are indicated.

FIG. 12 diagrams of nucleic acid constructs.

FIG. 13 shows photographs.

FIG. 14 shows Nε-(t-butyloxycarbonyl)-L-lysine (BocK) incorporation assay using Drosophila embryos.

FIG. 15 shows nucleic acid construct.

FIG. 16 shows nucleic acid construct.

FIG. 17 shows alkyne unnatural amino acids which may be used according to the present invention.

FIG. 18 shows a bar chart

FIG. 19 shows Drosophila expression constructs

FIG. 20 shows photographs

FIG. 21 shows photographs

FIG. 22 showsbar charts.

### EXAMPLES

To investigate the amber suppressor activity and potential orthogonality of the MbPylRS/tRNA_{CUA}^{Pyl} pair in S. cerevisiae we used MaV203:pGADGAL4(2TAG) cells.^{8,9} This yeast strain contains a GAL4 transcriptional activator gene bearing amber codons, is auxotrophic for histidine, and contains HIS3 and LacZ genes on GAL4 activated promoters. When a functional amber suppression system, such as the EeTyrRS/tRNA_{CUA}^{Pyl} pair,^{8,9} is transformed into this strain, full length GAL4 is produced, leading to activation of LacZ and HIS3 genes. Transcription of these genes allows cells to grow in the absence of histidine and turn blue in the presence of X-Gal.

Experimental Section

General methods N_{ε}-[(2-Propynyloxy)carbonyl]-L-lysine,⁵ N_{ε}-[(1-(6-nitrobenzo[d][1,3]dioxo1-5yl)ethoxy)carbonyl]-L-lysine² and N_{ε}-[(2-(3-methyl-3H-diazirin-3-yl)ethoxy)carbonyl]-L-lysine²³ were synthesized as previously reported. N_{ε}-Acetyl-L-lysine and N_{ε}-trifluoroacetyl-L-lysine were purchased from Bachem. Northern blot analysis Total RNA was purified from yeast cells using TRI reagent (Sigma) and ethanol precipitated. The RNA was denatured, separated on a 6% Novex TBE-urea gel (Invitrogen), blotted onto Biodyne B modified nylon membrane (Thermo Scientific) and crosslinked by UV fixation. The membrane was hybridized overnight at 55° C. with biotinylated probe 5′-GGAAACCCCGGGAATCTAACCCGGCTGAACGGATTTAGAG, which is specific for MbtDNA_{CUA}^{Pyl}. The hybridized probe was detected with North2South chemiluminescent hybridization and detection kit (Pierce). The number of cells was used to control the total amount of RNA loaded.

Phenotyping yeast cells Phenotyping was performed as described in Chin et al.,⁸ Briefly, S. cerevisiae MaV203 (Invitrogen) was transformed by the lithium acetate method with the pGADGAL4(2TAG) reporter, pMbPylRS and tDNA_{CUA}^{Pyl} constructs. Overnight cultures were serially diluted and replica plated onto selective media in the presence or absence of 2 mM N_{ε}-[(2-propynyloxy)carbonyl]-L-lysine (1). X-GAL assays were performed using the agarose overlay method.

Protein Expression, Purification, Western Blot Analysis and Mass Spectrometry

Appropriate selective medium±unnatural amino acid was inoculated with a stationary phase culture to give an O.D.₆₀₀˜0.2. Cultures were grown at 30° C. for 24-48 h. Proteins were extracted from yeast cells using Y-PER reagent (Thermo Scientific) containing complete, EDTA-free inhibitor cocktail (Roche). Clarified supernatants were separated by SDS-PAGE and western blots were performed using anti-His₆ (Qiagen). Human superoxide dismutase was purified using Ni²⁺-NTA resin (Qiagen) as previously described.²⁶ For expressions with N₆₈ acetyl-L-lysine (2), 20 mM nicotinamide was added to the culture and lysis buffers, and for expressions with trifluoroacetyl-L-lysine (3), 10 mM sodium butyrate was added to the culture and lysis buffers. Protein concentration was determined using the Biorad Protein Assay in comparison to IgG standard. Total mass analysis was performed on a LCT time-of-flight mass spectrometer with electrospray ionization (Micromass) with protein solutions in 20 mM ammonium bicarbonate and mixed 1:1 with 1% formic acid in 50% MeOH. Samples were injected at 10 and calibration was performed in positive ion mode using horse heart myoglobin. MS/MS analysis was performed on a LTQ-Orbitrap mass spectrometer on proteins samples that were in-gel digested with Glu-C (Roche).

The nucleotide sequences here below discussed are listed in a separate annex, Annex 1, at the end of the description.

Plasmid Construction

PCR reactions were carried out with Pfu or Pfu turbo polymerases (Stratagene) unless otherwise stated.

MbtDNA_{CUA}^{Pyl} cassettes: The MbtDNA_{CUA}^{Pyl} cassette was synthesized (Geneart). Site-directed mutagenesis was carried out using primers: P84/P85 (A box mutant: A11C/U24G/U15G); P82/P83 (B box mutant: A56C); P59/P60 (addition 3′-CCA). SNR52-MbtDNA_{CUA}^{Pyl}-SUP4 cassette: The tRNA cassette described in Wang et al.¹³ was synthesized (Geneart) with E. coli tDNA_{CUA}^{Pyl} replaced with MbtDNA_{CUA}^{Pyl}.

SNR6ᵤₚ-MbtDNA_{CUA}^{Pyl}-SNR6_{down} cassette: MbtDNA_{CUA}^{Pyl} was constructed from primers P88/P186. SNR6 upstream and downstream sequences were amplified from S. cereviasiae S288C genomic DNA with P183/184 and P187/P188 respectively. PCR fragments were assembled by overlap PCR.

Ile{TAT}LRI, Pro{TGG}FL and Asp{GTC}KR cassettes: MbtDNA_{CUA}^{Pyl} and SNR6 downstream sequences were amplified as above. The upstream sequences, as discussed in Dieci et al.¹⁵ were constructed with primers P189/P190 (Ile), P190 (pro) and P192 (Asp) and assembled with MbtDNA_{CUA}^{Pyl} and SNR6 downstream sequences by overlap PCR with Phusion polymerase (New England Biolabs).

SctDNA_{UCU}^{Arg}-MbtDNA_{CUA}^{Pyl} cassette: The cassette was built by consecutive overlapping PCR with ten primers (P164-P173) using Phusion polymerase. The SctDNA_{UCU}^{Arg}-MmtDNA_{CUA}^{Pyl} cassette was made by introducing the G3A mutation using primers P202/P203.

The tRNA cassettes were cloned into the XmaI/SpeI restriction sites of pRS426 (URA3, ATCC) using the AgeI/NheI restriction sites of the cassette.

Primers P217/P218 were cloned into the AgeI/NheI restriction sites of pEcTyrRS/EctRNA_{CUA}^{Tyr} to replace the EctDNA_{CUA}^{Tyr}. The codon-optimized gene for M. barkeri pyrrolysl-tRNA synthetase was cloned into EcoRI/NotI restriction sites of the resulting plasmid to replace E. coli tyrosyl-tRNA synthetase, giving plasmid pMbPylRS.

Primers P217/P218 were cloned into the AgeI/NheI restriction sites of pEcTyrRS/EctRNA_{CUA}^{Tyr 9} to replace the EctDNA_{CUA}^{Tyr}. The codon-optimized gene for M. barkeri pyrrolysl-tRNA synthetase was cloned into the EcoRI/NotI restriction sites of the resulting plasmid to replace E. coli tyrosyl-tRNA synthetase, giving plasmid pMbPylRS.

Variant Pyrrolysl-tRNA Synthetase

tRNA synthetases that aminoacylate MmtDNA_{CUA}^{Pyl} with N_{ε}-Acetyl-L-lysine (AcKRS3³), trifluoroacetyl-L-lysine (AcKRS2¹), N_{ε}-[(1-(6-nitrobenzo[d][1,3]dioxol-5yl)ethoxy)carbonyl]-L-lysine (PcKRS²) were created by transferring mutations identified in E. coli into the yeast codon-optimized MbPylRS gene. Primers P238-P241 and P287-P288 for the N_{ε}-Acetyl-L-lysyl synthetase and primers P244-P247 for N_{ε}-[(1-(6-nitrobenzo[d][1,3]dioxo1-5yl)ethoxy)carbonyl]-L-lysyl tRNA synthetase were used to amplify fragments from the codon-optimized MbPylS template, assembled by overlap PCR and recloned into the pMbPylRS vector. The tRNA synthetase that aminoacylates MmtDNA_{CUA}^{Pyl} with N_{ε}-Acetyl-L-lysine was created from N_{ε}Acetyl-L-lysine tRNA synthetase by site-directed mutagenesis using primers P242/P243.

Mutations from Wild-Type MbPylS:

| | |
|---|---|
| AcKRS3 | L266M/L270I/Y271F/L274A/C313F |
| PcKRS | M241F/A267S/Y271C/L274M |
| TfaKRS(AcKRS2) | L270I/Y271L/L274A/C313F |

### Example 1

### Wild Type MbPylRS/tRNA_{CUA}^{Pyl} in Yeast Cells

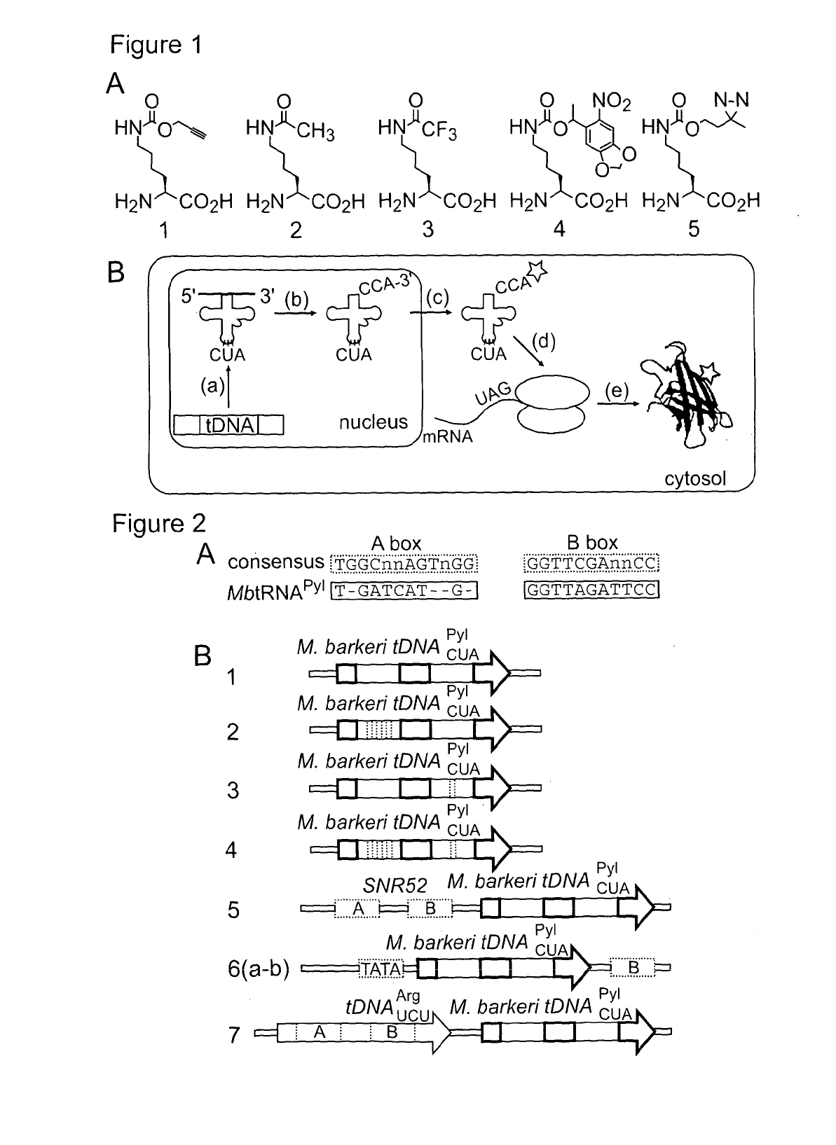

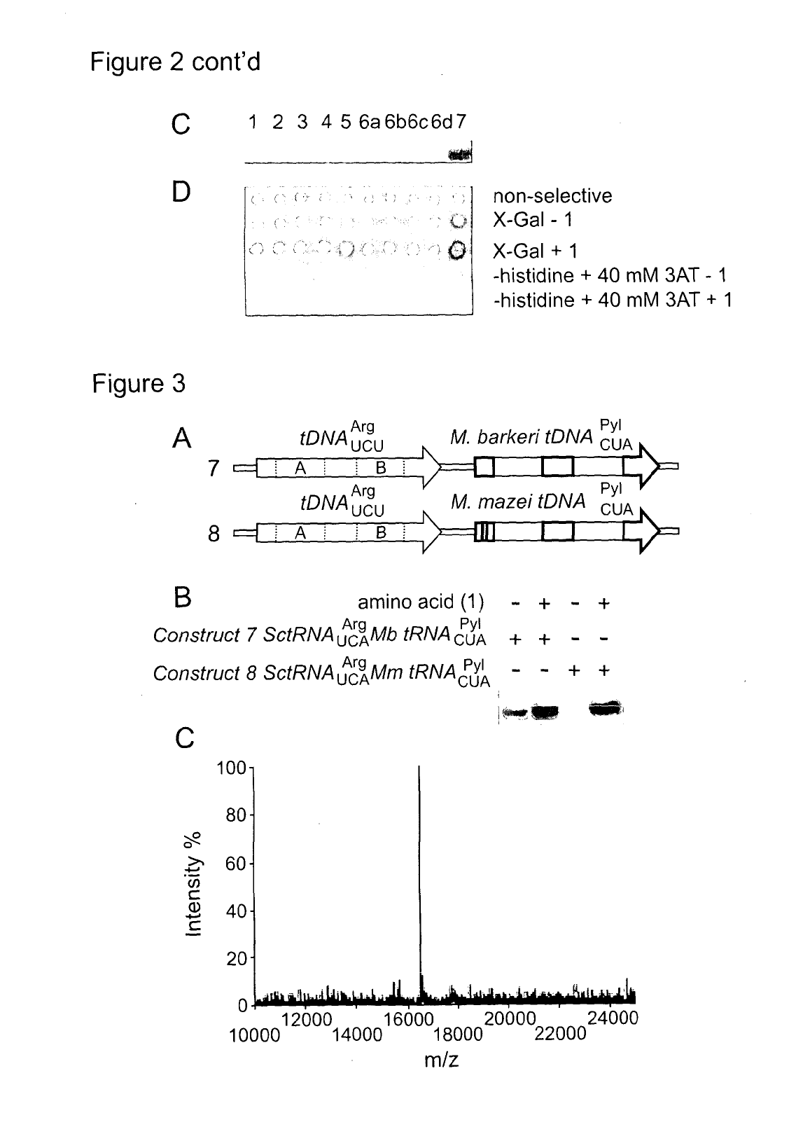

We replaced the functional EcTyrRS/tRNA_{CUA}^{Tyr} pair with the MbPylRS/MbtRNA_{CUA}^{Pyl} pair (FIG. 2B, construct 1) and supplemented with N_{ε}-[(2-propynyloxy)carbonyl]-L-lysine (1) (FIG. 1A, a known substrate for MbPylRS⁵) in MaV203:pGADGAL4(2TAG). These cells were unable to grow in media lacking histidine and did not turn blue in the presence of X-Gal, suggesting that this original construct is not functional (FIG. 2D). We demonstrated by western blot that the yeast codon-optimized MbPylRS was expressed in the cells (data not shown). However analysis of northern blots indicated that MbtRNA_{CUA}^{Pyl} was not transcribed from our initial construct (FIG. 2C). Since the EctRNA_{CUA}^{Tyr} gene contains the consensus A and B box RNA polymerase III promoter sequences that direct its transcription in yeast,¹¹ but MbtRNA_{CUA}^{Pyl} does not (FIG. 2A), it seemed likely that additional promoter elements would be required to direct the transcription of MbtRNA_{CUA}^{Pyl}.

### Example 2

### Promoter Elements Combined with Wild-Type MbPylRS/tRNA_{CUA}^{Pyl} in Yeast Cells

To address the challenge of creating new promoter elements to direct the transcription of MbtRNA_{CUA}^{Pyl}, we investigated strategies to introduce A and B box sequences into our tRNA expression construct. We first mutated the sequence of the MbtRNA_{CUA}^{Pyl} gene to contain either near-consensus A box sequences (A11C/U15G/T24G, FIG. 2B construct 2) or B box sequences (A56C, FIG. 2B construct 3). Northern blot analysis demonstrated that the A56C mutation in the B box, led to very low but detectable levels of the mutant MbtRNA_{CUA}^{Pyl} (Supplementary FIG. 1), while expression of the (A11C/U15G/T24G) mutant tRNA was not detectable by northern blot. However when the A56C mutant of MbtRNA_{CUA}^{Pyl} and MbPylRS were transferred to MaV203:pGADGAL4(2TAG) in the presence of 1 we did not observe phenotypes consistent with amber suppression (FIG. 2D). This implies that either the tRNA is transcribed but not correctly folded or processed, or that the mutation abolishes synthetase recognition. Combining the A and B box mutations (FIG. 2B construct 4) led to low levels of detectable tRNA production (Supplementary FIG. 1), but did not give phenotypes consistent with amber suppression (FIG. 2D).

Since enhancing the transcription of MbtRNA_{CUA}^{Pyl} by mutation of the A and B box sequences within the structural gene did not produce a functional amber suppressor, we next investigated the potential of constructs that might augment the transcription of MbtRNA_{CUA}^{Pyl} using extragenic sequences. The 5′-leader sequence of the yeast SNR52 primary transcript contains A and B box promoters that are post-transcriptionally removed to produce mature SNR52 snoRNA.¹² A previous report suggested that adding 5″-SNR52 and 3′-SUP4 flanking sequences to EctDNA_{CUA}^{Tyr} and EctDNA_{CUA}^{Leu} enhanced their amber suppression in yeast.¹³ When MbtRNA_{CUA}^{Tyr} was cloned between 5’-SNR52 and 3′-SUP4 flanking sequences (FIG. 2B construct 5) □ we could detect weak MbtRNA_{CUA}^{Pyl} transcription by northern blot (FIG. 2C), and when the cassette was transformed into MaV203:pGADGAL4(2TAG) containing MbPylRS and grown in the presence of 1, we observed blue coloration on X-Gal plates, but not growth in the absence of histidine in the presence of 40 mM 3AT (FIG. 2D). These data suggest that addition of extragenic A and B box sequences via the 5′-SNR52 and 3′-SUP4 flanking sequences can partially compensate for the absence of consensus A and B box sequences in MbtRNA_{CUA}^{Pyl}. However, since the EcTyrRS/tRNA_{CUA}^{Tyr} orthogonal pair supports growth on media lacking histidine and containing 40 mM 3AT^{8,9} but this system does not, we decided that the system was sub-optimal and opted to explore further extragenic sequences.

The yeast U6 (SNR6) gene assembles the same RNA polymerase III transcriptional machinery as tRNA genes but possesses an additional TATA-box promoter element 30 base pairs upstream of the transcription start site that binds TFIIIB.¹⁴ The TATA-box enables TFIIIC-independent RNA polymerase III recruitment and is proposed to overcome the large separation (240 bp) of the A and B-box promoter elements of this gene.¹⁵ Several yeast tRNAs, some of which contain large introns between the A and B-boxes, have TATA boxes that allow TFIIIC-independent RNA polymerase transcription.¹⁵ We reasoned that by incorporating the flanking sequences of these genes into our tRNA cassettes it may be possible to compensate for the poor A and B-box consensus of MbtRNA_{CUA}^{Pyl}. We created constructs where the 5′-flanking region of SNR6, Ile{TAT}LR1, Pro{TGG}FL and Asp{GTC}KR and the 3′-flanking region of SNR6 sandwich MbtRNA_{CUA}^{Pyl} (FIG. 2B, constructs 6a-d). We also added a consensus sequence¹⁶ found at the transcription start site of yeast tRNAs to the SNR6 construct. Northern blots revealed low level tRNA production from construct 6a (Supplementary FIG. 1). However we did not observe phenotypes consistent with amber suppression when any of these constructs were transformed into MaV203:pGADGAL4(2TAG) containing MbPylRS and grown in the presence of 1 (FIG. 2D). This data suggested that while these promoter elements may compensate for increases in the A and B box spacing they cannot efficiently compensate for defects in the A and B box sequence in MbtRNA_{CUA}^{Pyl}.

### Example 3

### tDNA_{UCU}^{Arg} as Promoter in Yeast Cells

We constructed a SctDNA_{UCU}^{Arg}-MbtDNA_{CUA}^{Pyl} cassette containing the natural 5′-, 3 and 10 base pair linker sequences (FIG. 2B, construct 7). Northern blot analysis revealed that MbtRNA_{CUA}^{Pyl} was transcribed from this construct much more efficiently than any other construct tested (FIG. 2C). When transformed into MaV203:pGADGAL4(2TAG) in the presence of MbPylRS and 1, the SctDNA_{UCU}^{Arg}-MbtDNA_{CUA}^{Pyl} cassette conferred survival on media lacking histidine and containing 40 mM 3AT, and produced the strongest blue color of any construct tested when incubated with X-Gal (FIG. 2D).

The tRNA constructs we discovered that are both transcribed, as judged by northern blot, and functional, as judged by phenotyping (constructs 5 and 7), showed amber suppression phenotypes even in the absence of added amino acid 1: construct 5 is blue on X-Gal in the presence and absence of 1, and construct 7 is blue in the presence and absence of 1 and grows on media lacking histidine and containing 3-aminotriazole (3AT) in the presence and absence of 1. These experiments revealed that MbtRNA_{CUA}^{Pyl} is not orthogonal in yeast.

### Example 4

### tRNA_{CUA}^{Pyl} is Orthogonal with Pyrrolysyl-tRNA Synthetase

To identify the molecular basis of the non-orthogonality of MbtDNA_{CUA}^{Pyl} we examined the sequence of MbtRNA_{CUA}^{Pyl} for nucleotides that match the positive identity elements within yeast tRNAs that are specifically recognized by yeast synthetases.²⁰ We realized that MbtRNA_{CUA}^{Pyl} contains an unusual G3-U70 base pair and wanted to test whether this caused the non-orthogonality in yeast cells. To test this hypothesis we expressed human superoxide dismutase (hSOD) bearing an amber codon at position 33 (from pC1 hSOD33TAG-His₆ in MJY125-derived strain SCY4²²). Expression of hSOD was dependent on the presence of SctDNA_{UCU}^{Arg}-MbtDNAP_{CUA}^{Pyl}, but did not decrease substantially in the absence of 1 (FIG. 3B), further confirming that the SctDNA_{UCU}^{Arg}-MbtDNA_{CUA}^{Pyl} cassette confers efficient amber suppression, which is not dependent on MbPylRS. ESI-MS of hSOD purified from expressions using MbPylRS/MbtRNA_{CUA}^{Pyl} in the absence of unnatural amino acid were consistent with the incorporation of alanine in response to the amber codon in hSOD33TAG (FIG. 3C), confirming our hypothesis on the molecular basis of MbtDNA_{CUA}^{CUA}^{Pyl} non-orthogonality. To create a MbtDNA_{CUA}^{Pyl} construct that is orthogonal in yeast we converted the G3-U70 base pair in MmtRNA_{CUA}^{CUA}^{Pyl} to A3-U70. This changes MbtRNA_{CUA}^{Pyl} to MmtRNA_{CUA}^{Pyl} (FIG. 3A, construct 8). Yeast containing MbPylRS/SctDNA_{UCU}^{Arg}-MmtDNA_{CUA}^{Pyl} produced full-length hSOD-His₆ from pC1SOD33TAG only in the presence of 1 (FIG. 3B). These experiments establish that functional MmtRNA_{CUA}^{Pyl} is produced from the dicistronic construct and is orthogonal in yeast.

### Example 5

### MbtRNA_{CUA}^{Pyl} is Orthogonal and Functions with a Wide Range of Unnatural Amino Acids

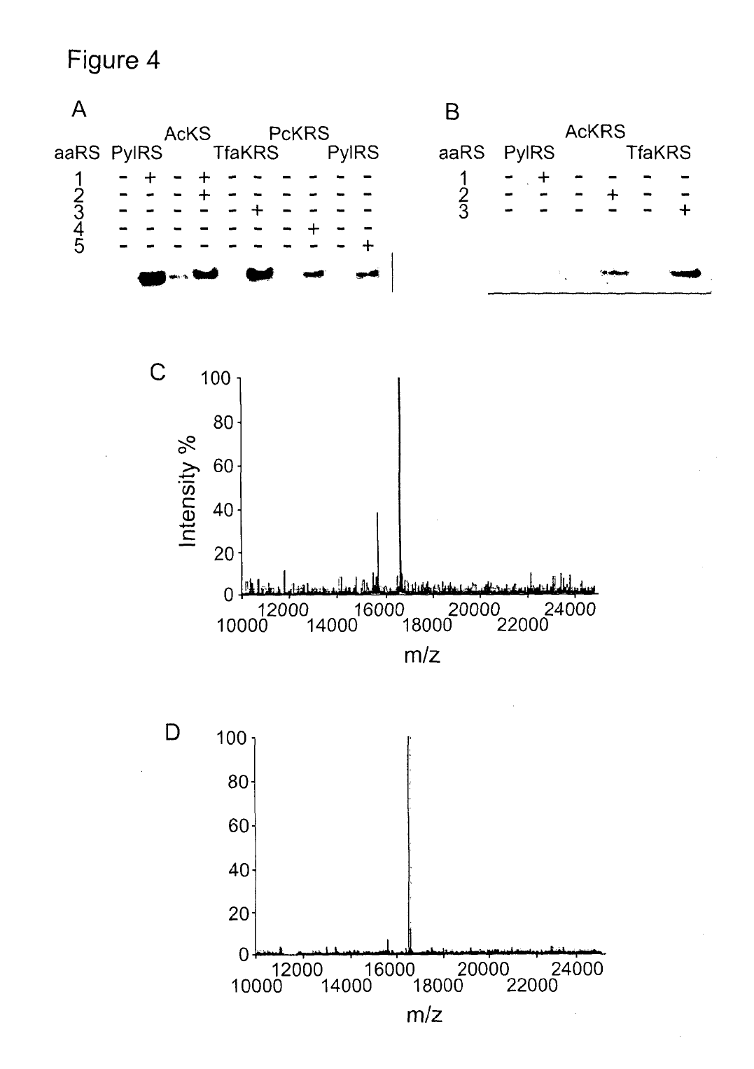

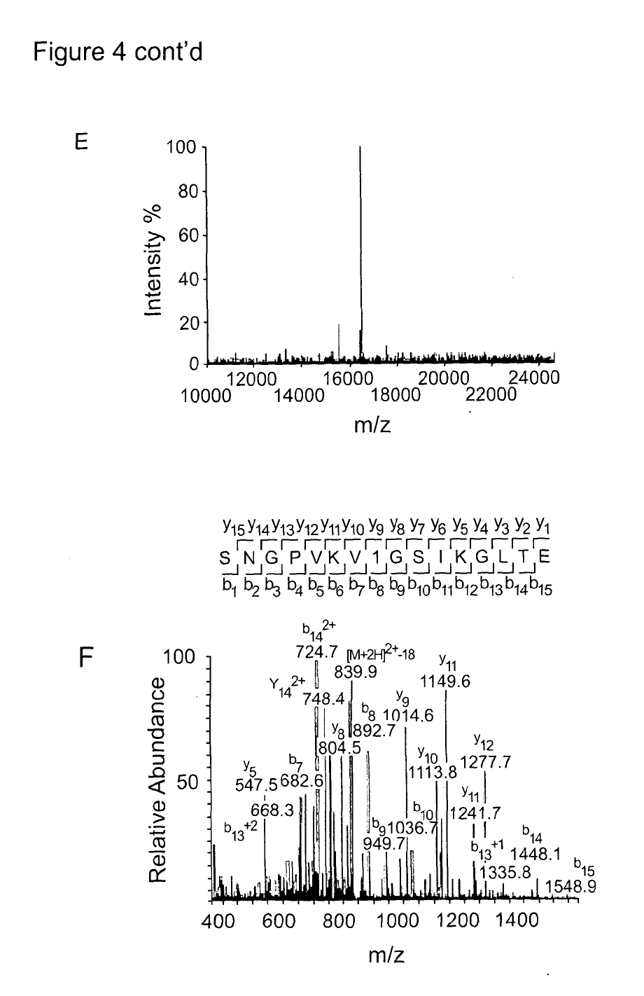

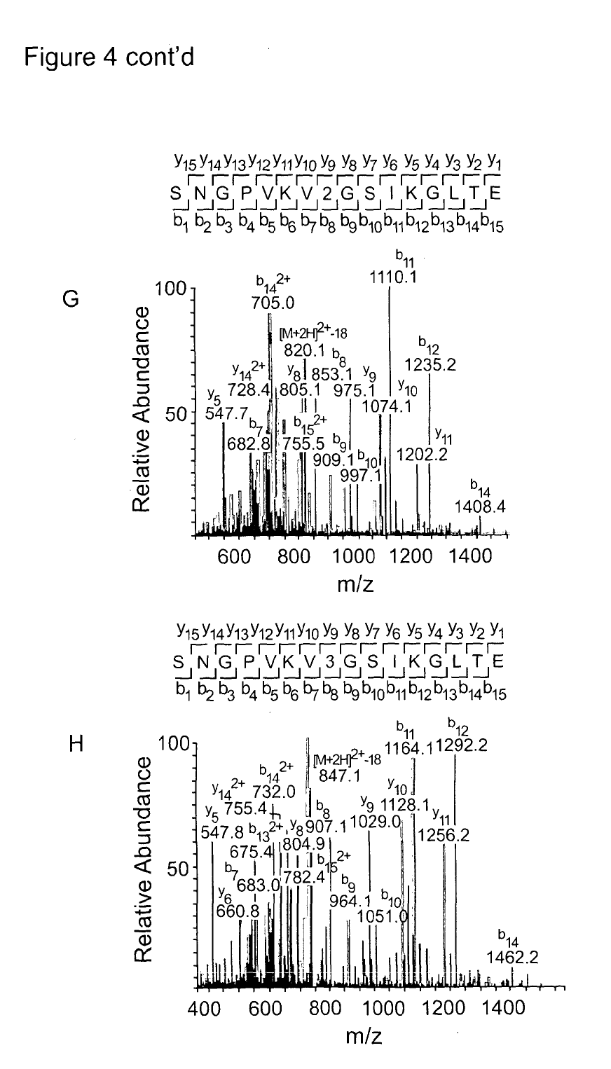

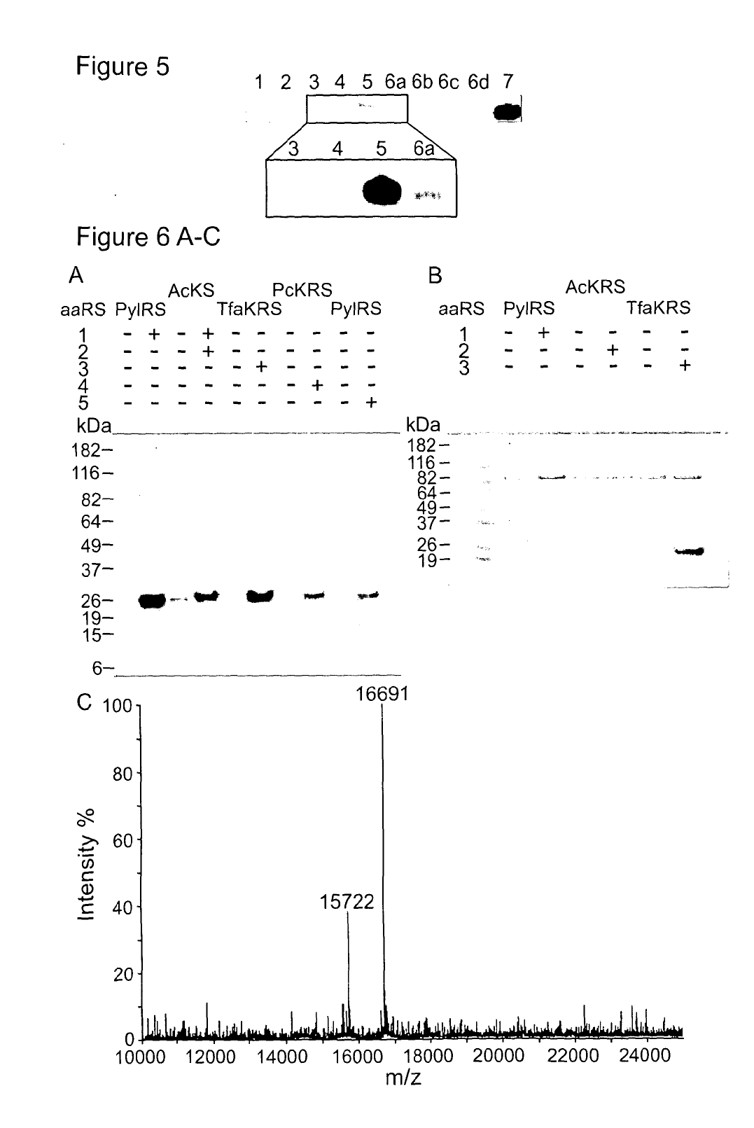

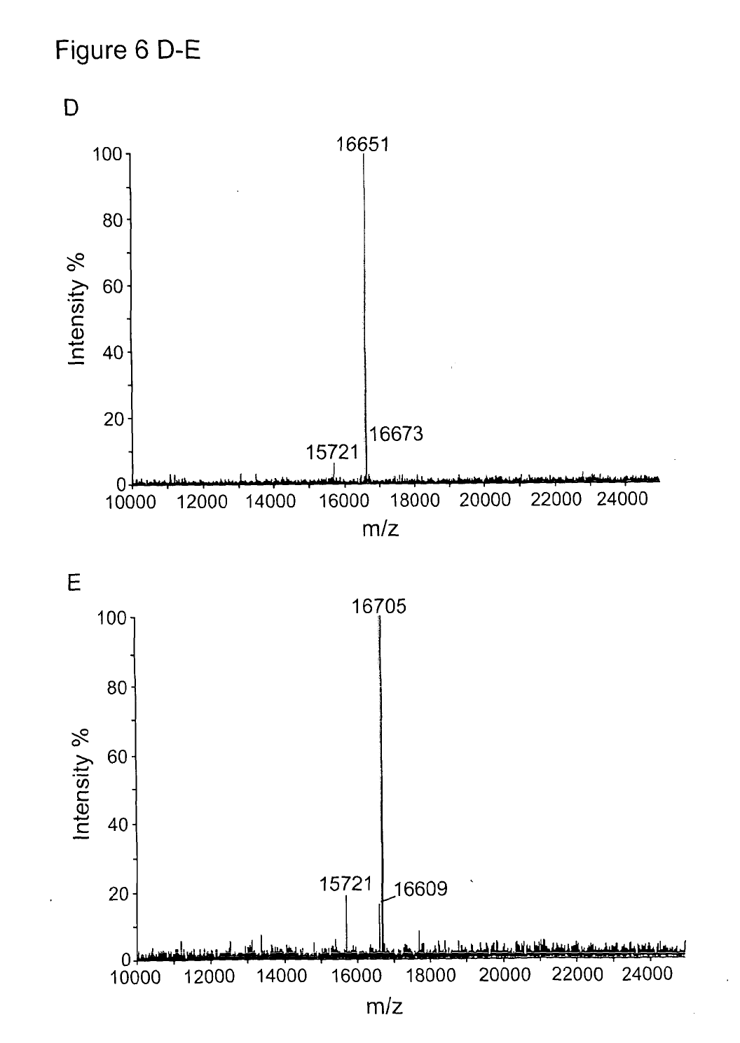

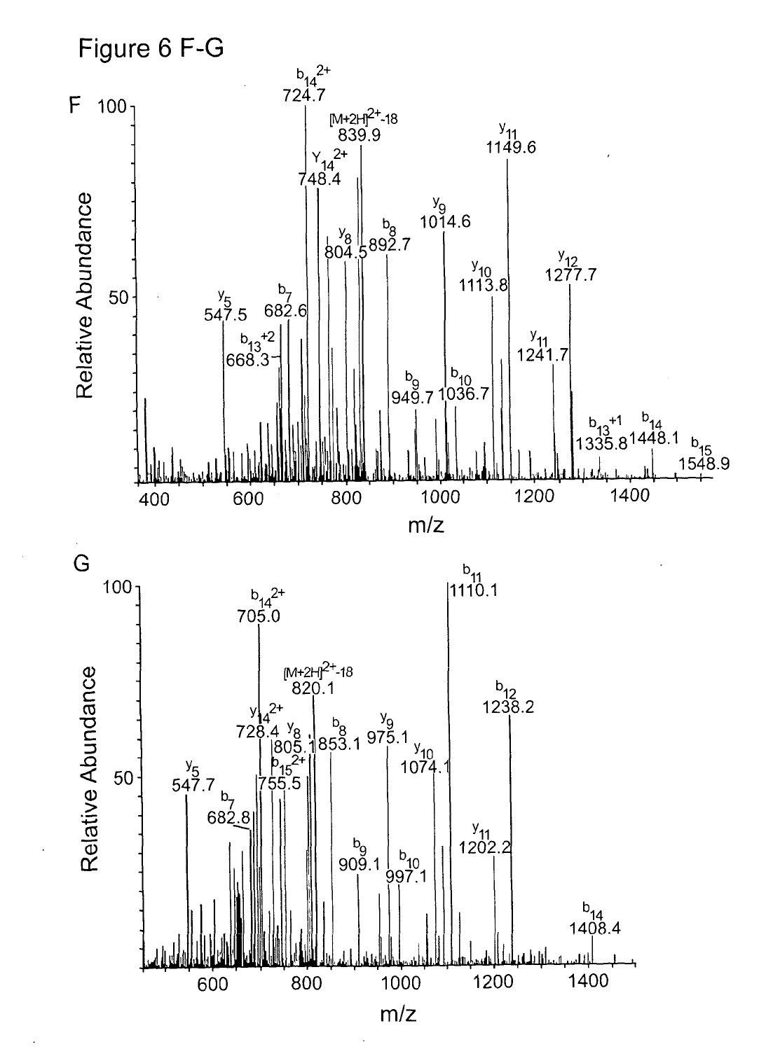

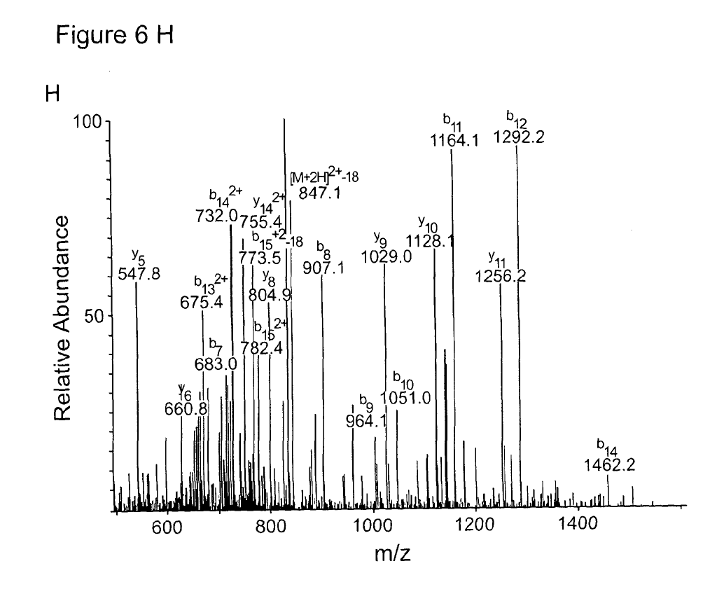

To begin to demonstrate the range of amino acids that can be incorporated in yeast using our approach, we incorporated the important post-translational modification N_{ε}-acetyl-L-lysine (2) and its analog N_{ε}-trifluoroacetyl-L-lysine (3), a photocaged lysine derivative N_{ε}-[(1-(6-nitrobenzo[d][1,3]dioxo1-5yl)ethoxy)carbonyl]-L-lysine (4), and photocrosslinker N_{ε}-[(2-(3-methyl-3H-diazirin-3-yl)ethoxy)carbonyl]-L-lysine (5) into hSOD-His₆ produced in S. cerevisiae (FIG. 4A) using MbPylRS and variants of MbPylRS we have previously evolved in E. coli.¹⁻³ While we have not specifically evolved a synthetase for N_{ε}-trifluoroacetyl-L-lysine, we have found that AcKRS2,¹ previously evolved for incorporating N_{ε}-acetyl-L-lysine, efficiently incorporates this amino acid. We demonstrated the incorporation of each amino acid by western blot (FIG. 4A). We carried out large-scale expression and purification of hSOD in the presence of 1, 2, and 3 (FIG. 4B), which unlike 4 and 5 are not photosensitive and are available in gram quantities, to further confirm the site and identity of amino acid incorporation by ESI-MS and MS/MS sequencing (FIG. 4C-H). We have demonstrated the specific incorporation of an amino acid into SOD in the presence of 4 and 5. In addition we have reported MS and MS/MS data for the incorporation of amino acids 4 and 5 into proteins in other organisms.^{2,23} However we have not yet obtained MS data directly in yeast for amino acids 4 and 5 and cannot rule out the possibility that an aspect of yeast metabolism—that is not conserved in either other eukaryotes or bacteria—leads to the selective post-translational modification of these amino acids in vivo. Purified hSOD yields were 30-100 μg per liter of yeast culture which is a similar to the 50 μg per liter yield reported for incorporating p-acetyl-L-phenylalanine into hSOD using the EcTyrRS/tRNA_{CUA}^{Try} pair in yeast.⁸

Conclusions

In summary we have solved the key challenges of producing a functional and orthogonal tRNA_{CUA}^{Pyl} in yeast. We have discovered an MbPylRS/tRNA_{CUA}^{Pyl} pair that is orthogonal in yeast, and described a simple system through which variant MbPylRS/tRNA_{CUA}^{Pyl} pairs created in E. coli can be transplanted to expand the genetic code of yeast for a wide range of unnatural amino acids. Using our approach we have incorporated the alkyne-containing amino acid N_{ε}-[(2-propynyloxy)carbonyl]-L-lysine (1), an important post-translationally modified amino acid N-acetyl-L-lysine (2) and an analog of N_{ε}-acetyl-L-lysine, trifluoroacetyl-L-lysine (3), a photocaged lysine derivative N_{ε}-[(1-(6-nitrobenzo[d][1,3]dioxo1-5yl)ethoxy)carbonyl]-L-lysine (4), and a photocrosslinker N_{ε}-[(2-(3-methyl -3H-diazirin-3-yl)ethoxy)carbonyl]-L-lysine (5) into proteins in yeast. Amino acid 1 may be used for bio-orthogonal 3+2 cycloadditions in yeast proteins.²⁴ Amino acid 2 may be used for producing acetylated proteins directly in yeast and synthetically controlling processes normally regulated by acetylation in yeast. Amino acid 3 is a very poor substrate for sirtuins but not HDACs²⁵ and should allow us to install irreversible acetylation at sites directly regulated by sirtuins in vivo. It should allow us to probe the deacetylases that act on a given site in a protein. Amino acid 4 is a photocaged lysine with demonstrated utility for controlling protein function in eukaryotic cells² and we anticipate that genetically-encoded photocontrol to of proteins in yeast will be a powerful approach for gaining a temporal and spatial understanding of cellular processes. Amino acid 5 is a photocrosslinking amino acid with demonstrated utility mapping protein interactions in E. coli²³ and we believe that this will find wide utility in mapping protein-protein interactions in yeast. Given the growing list of amino acids that can be incorporated using MbPylRS and its variants,¹⁻⁶ we anticipate that our approach will allow the introduction of a wide range of chemical functional groups into yeast. Finally, the strategies we have explored for creating and expressing heterologous, orthogonal tRNAs in yeast, may be useful for improving other orthogonal aminoacyl-tRNA synthetase/tRNA_{CUA} pairs systems.⁸⁻¹⁰

### REFERENCES

(1) Neumann, H.; Peak-Chew, S. Y.; Chin, J. W. Nat. Chem. Biol. 2008, 4, 232.
(2) Gautier, A.; Nguyen, D. P.; Lusic, H.; An, W.; Deiters, A.; Chin, J. W. J. Am. Chem. Soc. 2010, 132, 4086.
(3) Neumann, H.; Hancock, S. M.; Buning, R.; Routh, A.; Chapman, L.; Somers, J.; Owen-Hughes, T.; van Noort, J.; Rhodes, D.; Chin, J. W. Mol. Cell. 2009, 36, 153.
(4) (a) Zhao, S.; Xu, W.; Jiang, W.; Yu, W.; Lin, Y.; Zhang, T.; Yao, J.; Zhou, L.; Zeng, Y.; Li, H.; Li, Y.; Shi, J.; An, W.; Hancock, S. M.; He, F.; Qin, L.; Chin, J.; Yang, P.; Chen, X.; Lei, Q.; Xiong, Y.; Guan, K. L. Science 2010, 327, 1000; (b) Nguyen, D. P.; Garcia Alai, M. M.; Kapadnis, P. B.; Neumann, H.; Chin, J. W. J. Am. Chem. Soc. 2009, 131, 14194; (c) Fekner, T.; Li, X.; Lee, M. M.; Chan, M. K. Angew. Chem., Int. Ed. 2009, 48, 1633; (d) Li, W. T.; Mahapatra, A.; Longstaff, D. G.; Bechtel, J.; Zhao, G.; Kang, P. T.; Chan, M. K.; Krzycki, J. A. J. Mol. Biol. 2009, 385, 1156; (e) Li, X.; Fekner, T.; Ottesen, J. J.; Chan, M. K. Angew. Chem., Int. Ed. 2009, 48, 9184; (f) Yanagisawa, T.; Ishii, R.; Fukunaga, R.; Kobayashi, T.; Sakamoto, K.; Yokoyama, S. Chem. Biol. 2008, 15, 1187.
(5) Nguyen, D. P.; Lusic, H.; Neumann, H.; Kapadnis, P. B.; Deiters, A.; Chin, J. W. J. Am. Chem. Soc. 2009, 131, 8720.
(6) Chen, P. R.; Groff, D.; Guo, J.; Ou, W.; Cellitti, S.; Geierstanger, B. H.; Schultz, P. G. Angew. Chem. Int. Ed. 2009, 48, 4052.
(7) Mukai, T.; Kobayashi, T.; Hino, N.; Yanagisawa, T.; Sakamoto, K.; Yokoyama, S. Biochem. Biophys. Res. Commun. 2008, 371, 818.
(8) Chin, J. W.; Cropp, T. A.; Anderson, J. C.; Mukherji, M.; Zhang, Z.; Schultz, P. G. Science 2003, 301, 964.
(9) Chin, J. W.; Cropp, T. A.; Chu, S.; Meggers, E.; Schultz, P. G. Chem. Biol. 2003, 10, 511.
(10) Wu, N.; Deiters, A.; Cropp, T. A.; King, D.; Schultz, P. G. J. Am. Chem. Soc. 2004, 126, 14306.
(11) Edwards, H.; Schimmel, P. Mol. Cell. Biol. 1990, 10, 1633.
(12) Guffanti, E.; Ferrari, R.; Preti, M.; Forloni, M.; Harismendy, O.; Lefebvre, O.; Dieci, G. J. Biol. Chem. 2006, 281, 23945.
(13) Wang, Q.; Wang, L. J. Am. Chem. Soc. 2008, 130, 6066.
(14) Brow, D. A.; Guthrie, C. Genes Dev. 1990, 4, 1345.
(15) Dieci, G.; Percudani, R.; Giuliodori, S.; Bottarelli, L.; Ottonello, S. J. Mol. Biol. 2000, 299, 601.
(16) Raymond, K. C.; Raymond, G. J.; Johnson, J. D. EMBO J. 1985, 4, 2649.
(17) Schmidt, O.; Mao, J.; Ogden, R.; Beckmann, J.; Sakano, H.; Abelson, J.; Söll, D. Nature 1980, 287, 750.
(18) (a) Kjellin-Straby, K.; Engelke, D. R.; Abelson, J. DNA 1984, 3, 167; (b) Reyes, V. M.; Newman, A.; Abelson, J.Mol. Cell. Biol. 1986, 6, 2436.
(19) (a) Francis, M. A.; Rajbhandary, U. L. Mol. Cell. Biol. 1990, 10, 4486; (b) Straby, K. B. Nucleic Acids Res. 1988, 16, 2841.
(20) Giege, R.; Sissler, M.; Florentz, C. Nucleic Acids Res. 1998, 26, 5017.
(2 1) (a) Hou, Y. M.; Schimmel, P. Nature 1988, 333, 140; (b) McClain, W. H.; Foss, K. Science 1988, 240, 793.
(22) Summerer, D.; Chen, S.; Wu, N.; Deiters, A.; Chin, J. W.; Schultz, P. G. Proc. Natl. Acad. Sci. USA 2006, 103, 9785.
(23) Chou, C.; Upretya, R.; Davis, L.; Chin, J. W.; Deiters, A. Submitted 2010.
(24) (a) Kolb, H. C.; Finn, M. G.; Sharpless, K. B. Angew. Chem., Int. Ed. 2001, 40, 2004; (b) Meldal, M.; Tornoe, C. W. Chem. Rev. 2008, 108, 2952.
(25) (a) Smith, B. C.; Denu, J. M. J. Biol. Chem. 2007, 282, 37256; (b) Smith, B. C.; Hallows, W. C.; Denu, J. M. Chem. Biol. 2008, 15, 1002.
(26) Chen, S.; Schultz, P. G.; Brock, A. J. Mol. Biol. 2007, 371, 112.

### Example 6

### Multicellular Eukaryotes

Genetic code expansion methods, utilizing orthogonal aminoacyl-tRNA synthetase/tRNA_{CUA} pairs, have facilitated the site-specific incorporation of unnatural amino acids into proteins in E. coli, in yeast and in mammalian cells¹⁻⁶. The application of unnatural amino acid mutagenesis to the production of recombinant proteins allows access to modified proteins, including proteins bearing defined post-translational modifications, for structural biology, enzymology, and single molecule studies⁶⁻¹³. The genetically encoded incorporation of photocaged amino acids in living cells allows the photo-control of protein interactions, protein localization, enzymatic activity and signaling^{3,14-16}, while the incorporation of photocrosslinking amino acids allows the mapping of weak or transient protein interactions, including those in membranes, that are hard to trap by traditional non-covalent approaches^{14,17-20}, and the incorporation of bio-orthogonal chemical handles and biophysical probes are providing new approaches for imaging and spectroscopy^{21,22}. Despite these advances, genetic code expansion methods are currently limited to unicellular systems.

Approaches to site-specifically incorporating unnatural amino acids into proteins in multicellular organisms may ultimately facilitate the extension of the approaches developed for the real time, molecular dissection of biological process inside cells^{3,14-16,23} to the study of complex processes that require interactions between cells in an organism, such as development and neural processing.

C. elegans is our first target for a multicellular genetic code expansion. The genome of C. elegans is sequenced²⁴ and the lineage of every cell during embryogenesis and post-embryonic development has been mapped in this organism^{25,26} , which is invaluable in understanding mutant phenoypes at the cellular level. The organism has around 1000 somatic cells that make up a variety of tissues including muscles, nerves and intestines. The entire organism is transparent at every stage of life, making it possible to visualize expression in individual cells using fluorescent proteins. This will facilitate light mediated intervention in biological processes using genetically encoded photo-responsive amino acids, including photocrosslinkers and photcaged amino acids. Many biochemical and signalling pathways involved in disease are conserved between C. elegans and humans, which makes C. elegans an important organism for identifying the molecular mechanisms of disease²⁷. Moreover, C. elegans is the only multicellular organism where amber suppressors have been isolated and introduced into the germ line by classical genetics approaches²⁸⁻³¹, and suppression efficiencies exceeding 30% have been reported³². These observations suggest that amber suppression is not problematic for the organism through development and reproduction.

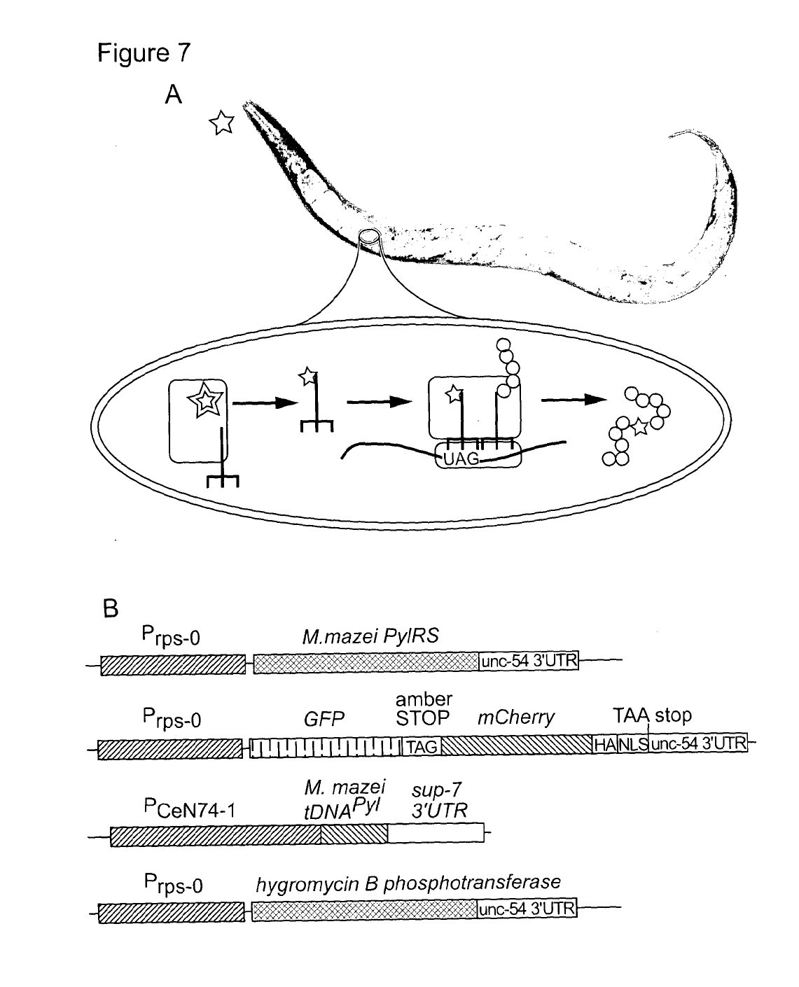

The site-specific incorporation of unnatural amino acids into target proteins poses a number of challenges (FIG. 7): We require an orthogonal amber suppressor tRNA, that is correctly transcribed processed, modified and exported to the cytoplasm of the cell, an orthogonal aaRS that can uniquely aminoacylate the orthogonal tRNA in the cytoplasm, and an mRNA encoding a gene of interest bearing an amber codon that directs amino acid incorporation. In addition, we need to combat any effects of nonsense-mediated decay that may destroy transcripts bearing amber codons and limit expression of proteins bearing unnatural amino acids. The site specific incorporation of unnatural amino acids in an animal poses additional challenges, since each of the translational components must be present in the same cell or cells within the organism to effect genetic code expansion, and we need to ensure that the unnatural amino acids are taken up by the animal and are available, within the cytoplasm of its cells, for protein translation.

We created a reporter for amber suppression (Prps-0::mGFP-TAG-mcherry-HA-NLS) in which a 5′ mGFP is separated from a 3′ mCherry gene by a linker region containing an amber stop codon (FIG. 7). The ribosomal protein promoter (Prps-0) in this construct drives expression in most cells in the worm³³, the HA tag allows detection of expression by anti-HA antibodies, the nuclear localization sequence (NLS) concentrates fluorescence in the nucleus and the unc-54 3′ untranslated region (UTR) stabilizes the mRNA throughout the worm. We injected this reporter into C. elegans using a construct carrying wild type lin-15B as a selection marker in a lin-15B(n765) genetic background³⁴. We observed that the transmission frequency of the transgenic extra-chromosomal arrays to offspring was low (20-30%). This resulted in C. elegans populations where a majority of animals did not carry the transgenes. Moreover we observed that the GFP signal in worms carrying the reporter was much weaker than the GFP signal produced from a simple GFP gene.

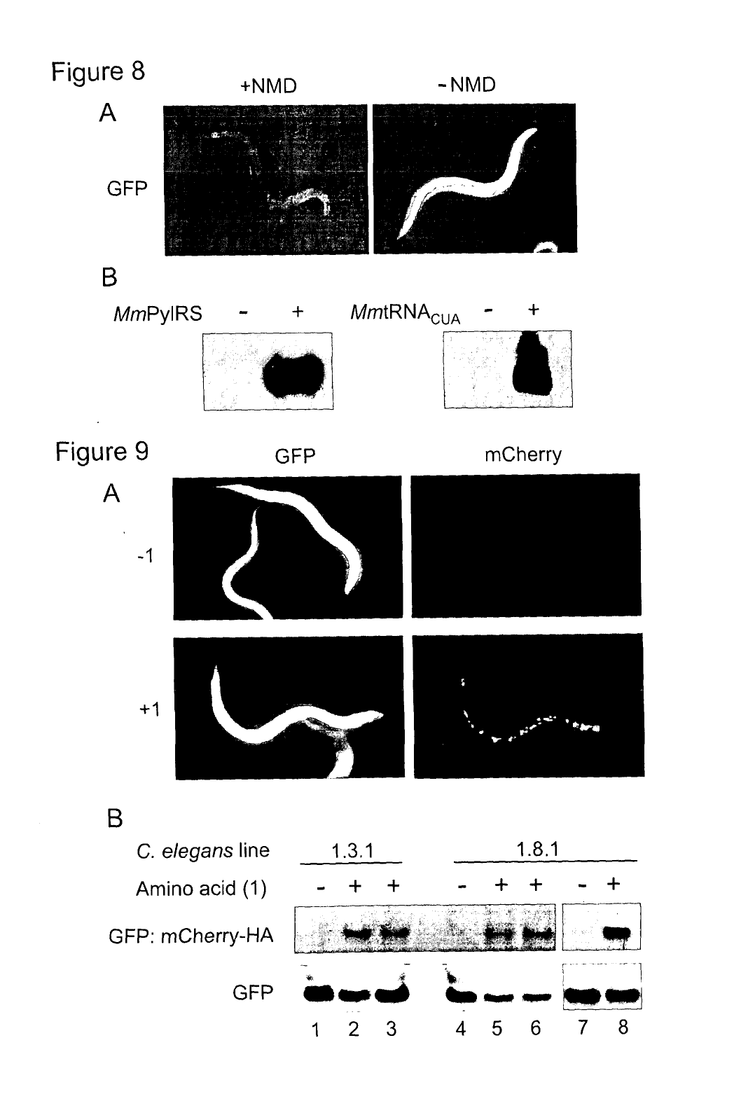

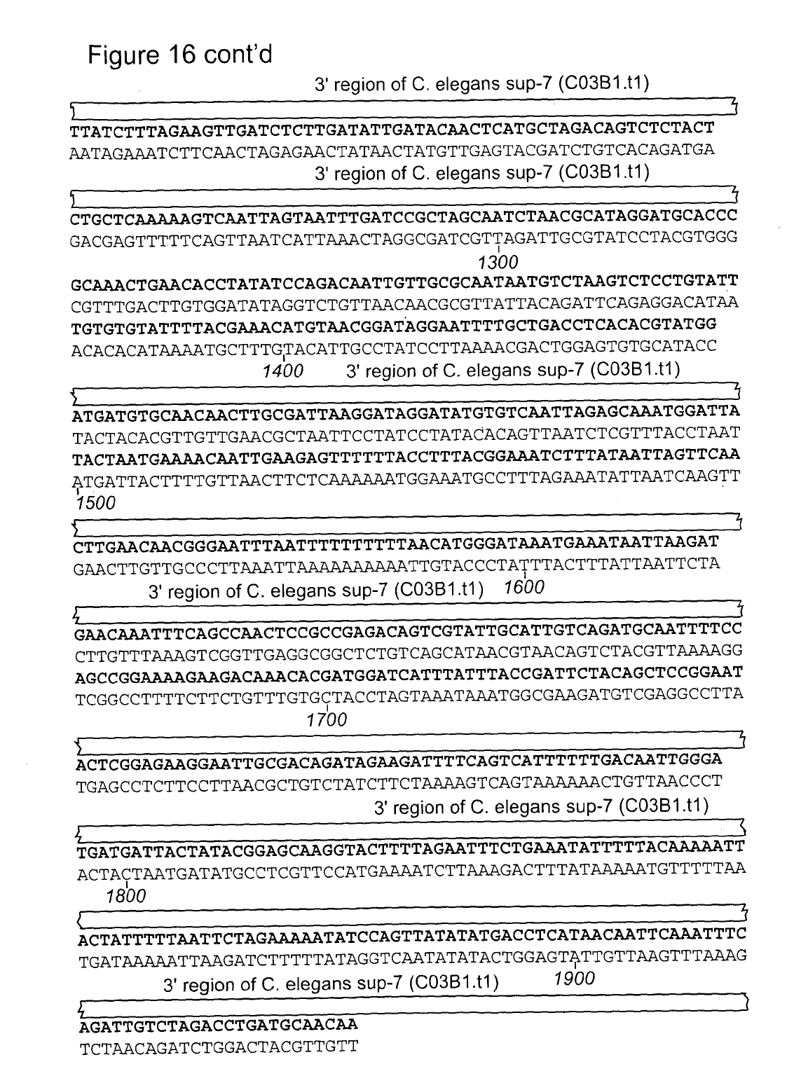

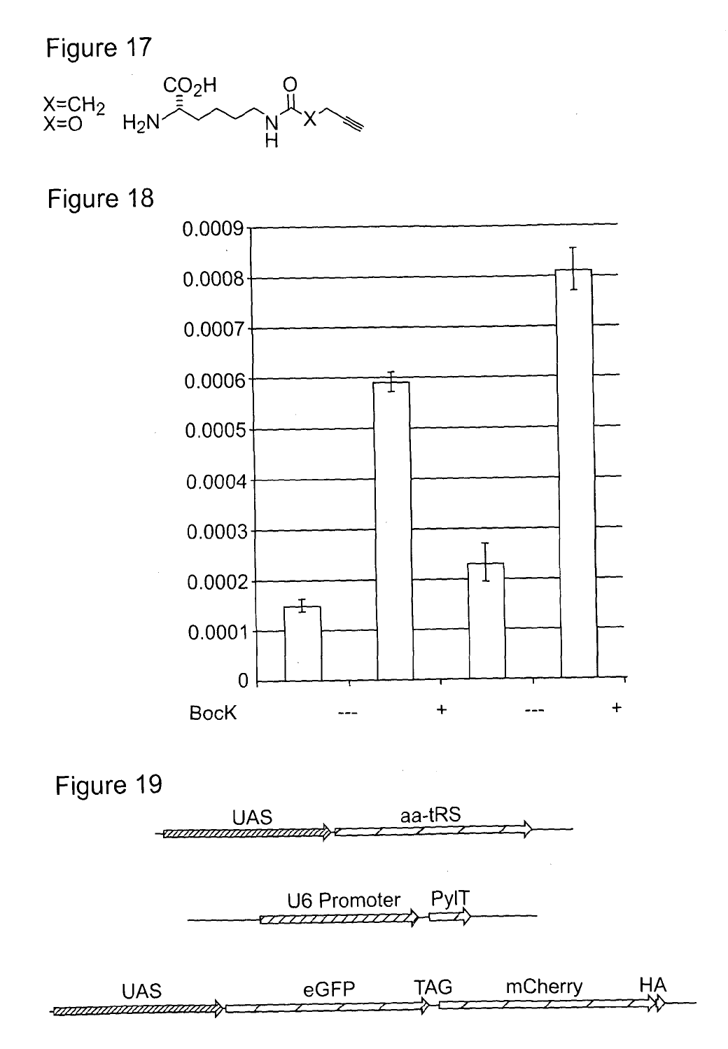

We reasoned that the low GFP expression was likely due to the degradation of reporter mRNA through nonsense mediated decay (NMD), a surveillance mechanism present in eukaryotic cells that is responsible for detecting and destroying transcripts with premature stop codons^{35,36}. When we crossed worms expressing the reporter with smg-2(e2008) worms that are deficient in NMD^{35,37}, but otherwise healthy, we observed a striking increase of GFP signal (FIG. 8 & FIG. 10). While we see a strong GFP signal in worms transformed with the reporter we do not observe any mCherry fluorescence, demonstrating that the reporter is functional and that the worms do not contain endogenous amber suppressors. Based on these observations we constructed all subsequent transgenic lines using the smg-2(e2008) worms.

To address the problem of low transmission levels we tested transformation markers that use a gene conferring resistance to specific antibiotics. Recent reports use puromycin³⁸ and G-418³⁹ resistance genes respectively for antibiotic based selection in worms. However, puromycin efficiently kills wild type animals only in the presence of the permeabilizing detergent, Triton X-100, and G-418 does not kill all wild type worms in a population. We therefore investigated a further antibiotic, hygromycin B⁴⁰, which is used for selection in eukaryotic cell culture, but has not been used as a selectable marker in C. elegans. We found that hygromycin B (0.5 mg/ml) kills 100% of wild type worms without the addition of Triton X-100 (data not shown). When the hygromycin B phosphotransferase gene (hpt) fused to the rps-0 promoter (Prps-0::hpt) was injected into worms it conferred resistance to the antibiotic. Using this approach we were able to isolate transgenic lines that appear to have transmission rates of 100% in the presence of hygromycin B (data not shown). In all subsequent experiments we used hygromycin B resistance as a marker for introducing DNA constructs into C. elegans.

Three aminoacyl-tRNA synthetase/tRNA_{CUA} pairs (EcTyrRS/tRNA_{CUA}, EcLeuRS/tRNA_{CUA} and PylRS/tRNA_{CUA} from methanosarcina species) are orthogonal in eukaryotic cells and have been used to incorporate unnatural amino acids⁴¹. We and other have demonstrated that the PylRS/tRNA_{CUA} pairs from methanosarcina species including M. barkeri (Mb) and M. mazei (Mm), which naturally uses pyrrolysine, can be used to incorporate a range of unnatural amino acids, including Nε-(1-butyloxycarbonyl)-L-lysine (6)¹³. Moreover, the PylRS/tRNA_{CUA} pairs can be evolved in E. coli to recognize new amino acids⁶, and then be transplanted into eukaryotic cells⁴¹. This is in contrast to the other pairs that need to be evolved for new amino acid specificity directly in a eukaryotic host. Since the library construction methods for synthetase evolution are straightforward in E. coli it is especially attractive to develop the PylRS/tRNA_{CUA} system for incorporating unnatural amino acids in animals.

To express MmPylRS from an RNA Polymerase II (Pol II) promoter we created Prps-0::FLAG-MmPylRS, in which Prps-0 directs expression throughout the animal, the FLAG tag allows the expression of PylRS to be detected by western blot. Western blots demonstrate that the synthetase is expressed in the worm (FIG. 8 and FIG. 10).

MmtRNA_{CUA} requires RNA polymerase III transcription. Transcription of eukaryotic tRNAs by RNAP III is directed by A and B box sequences that are internal to the tRNA gene. Theses sequences are not present in the orthogonal MmtRNA_{CUA} gene and it is challenging to introduce such sites without disrupting the three dimensional structure and functionality of the mature tRNA⁴. We therefore investigated extragenic RNA polymerase III promoters for the transcription of MmtRNA_{CUA}. To direct the transcription of MmtRNA_{CUA} we created PCeN74-1::MmPylT::sup-7 3′, in which the selected Pol III promoter, derived from the stem-bulge non coding RNA (ncRNA) CeN74-1 is fused to the 5′ end of the MmtRNA_{CUA} gene and transcription of the tRNA is terminated by the region found immediately 3′ of the sup-7 C. elegans tryptophanyl tRNA gene. We chose the CeN74-1 promoter, since it shows a high level of expression in adult animals, and some expression in larval stages^{42,43}; we reasoned that these properties would enable us to more efficiently screen for cells or animals expressing a functional tRNA, since worms are in the adult stage for up to several weeks but are only in the larval stages for a short period. Northern blots, using a probe specific for MmtRNA_{CUA}⁴, demonstrate that the tRNA is efficiently produced from this promoter in C. elegans (FIG. 8 & FIG. 10).

We constructed lines containing all genetic components by biolistic bombardment⁴⁴ of sing-2(e2008) worms with plasmids encoding the reporter, synthetase, tRNA and hygromycin B phosphotransferase gene (Prps-0::mGFP-TAG-mcherry-HA-NLS, Prps-0::FLAG-MmPylRS, PCeN74-1::MmPylT, Prps-0::hpt). The transformants were grown on plates supplemented with hygromycin B for 2 weeks to kill off all non transgenic worms, resulting in populations where all worms contained the extra-chromosomal transgenic array Ex1[Prps-0::mGFP-TAG-mcherry-HA-NLS; Prps-0::FLAG-MmPylRS; PCeN74-1::MmPylT; Prps-0::hpt]. Surviving worms were grown on 5 mM (6) and inspected by fluorescence microscopy for the presence of mCherry in the nucleus of cells within the worm. This step allowed us to select for animals expressing the reporter as well as functional MmPylRS and MmtRNA_{CUA}.

We examined several thousand worms and saw a few (1 to 5) mCherry positive worms per hundred worms examined. Individual worms showed mCherry expression in different tissues, including intestinal cells, pharyngeal cells, neurons and body wall muscle. The mosaicism of expression from these extrachromosomal arrays is well documented and results either from loss of the array during mitosis or partial or complete silencing of the array.

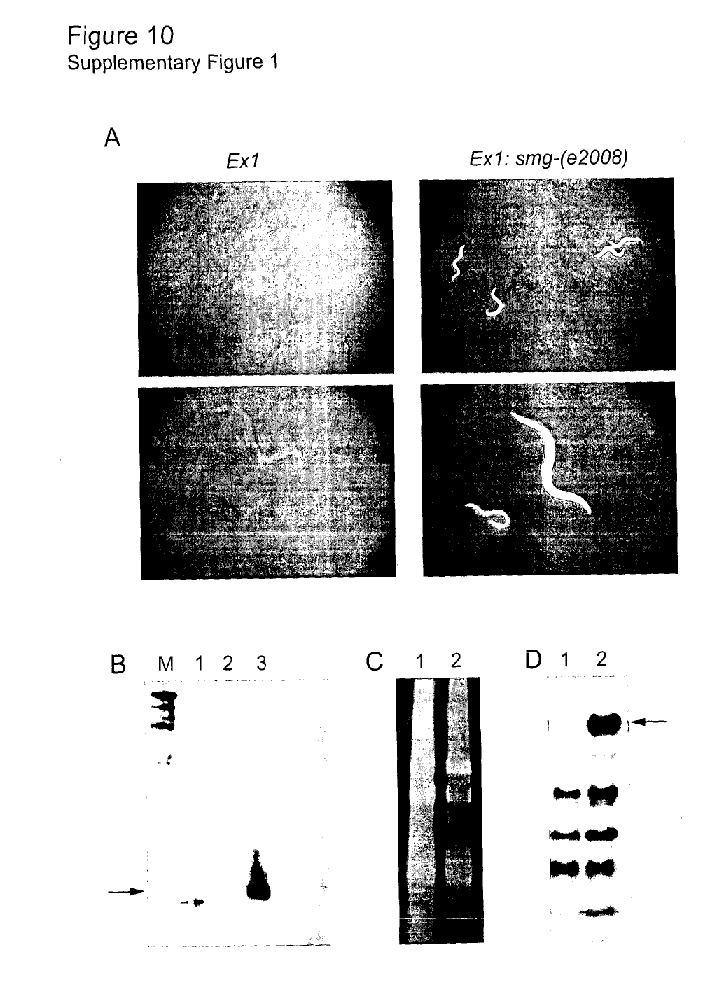

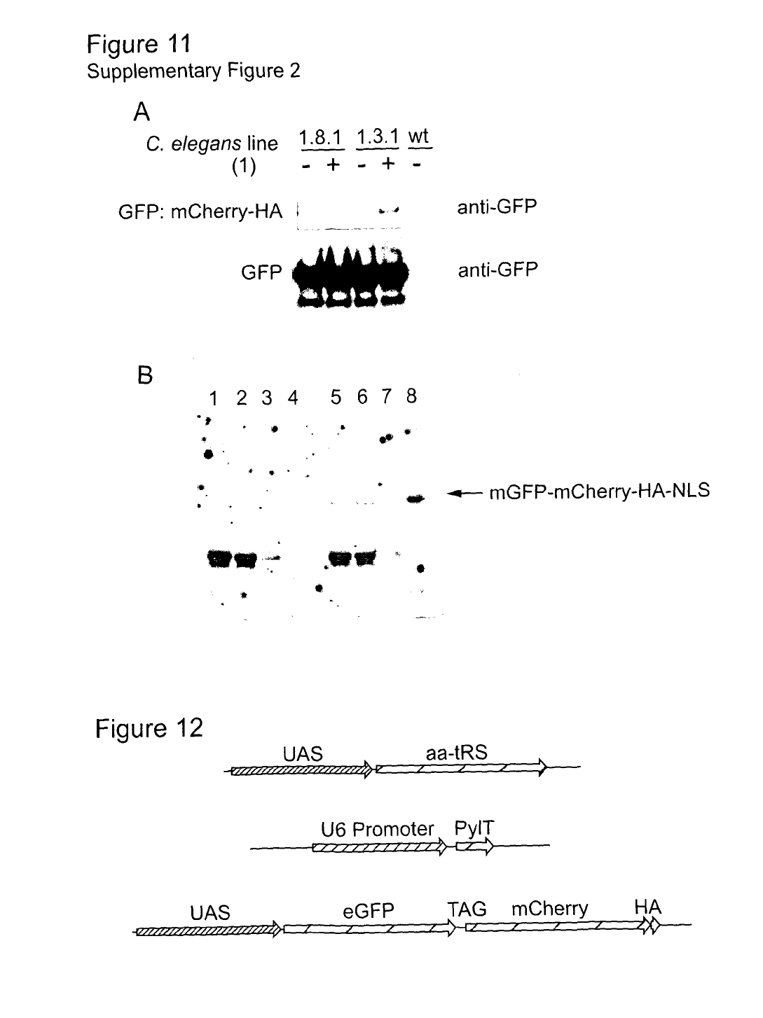

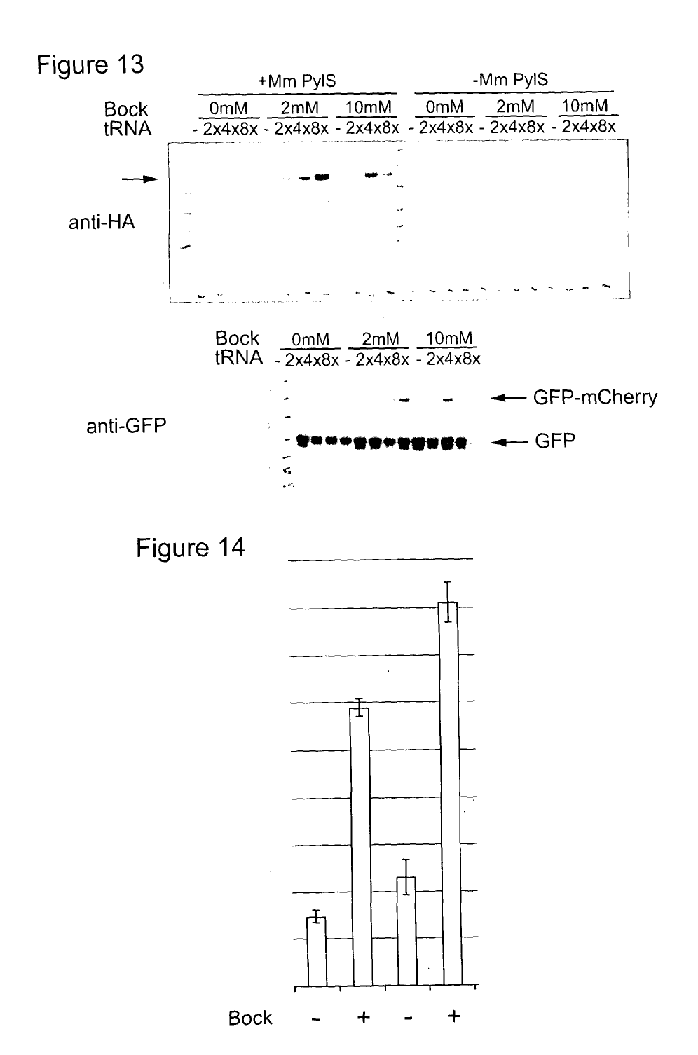

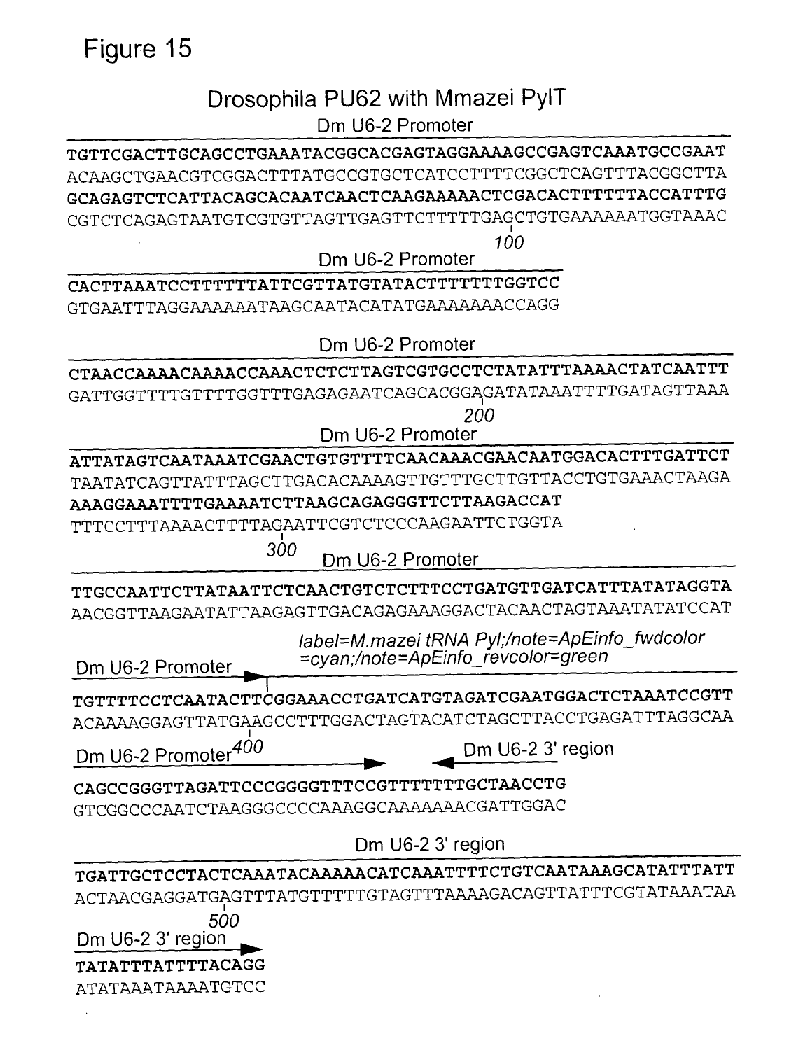

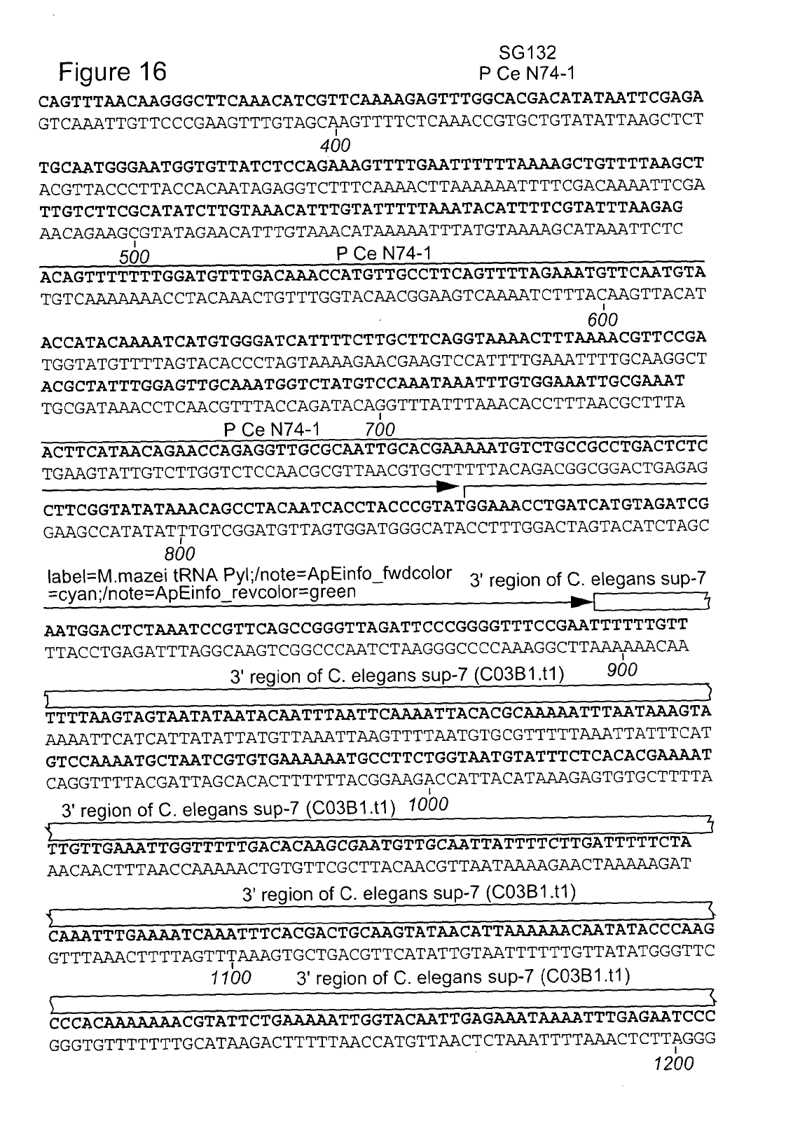

We singled out 13 mCherry positive worms and grew them in the absence of (6) and the presence of hygromycinB, to select for inheritance of the array in the resulting lines. We examined these lines for mCherry fluorescence in the presence and absence of (6). While all lines selected showed amino acid dependent mCherry fluorescence, we focused in subsequent experiments on two lines (1.3.1 and 1.8.1). These lines were singled out from distinct plates, and showed the strongest mCherry fluorescence in the presence of amino acid (6). In the absence of amino acid (6) we did not find any worms expressing mCherry, in the several thousand animals we screened by fluorescence microscopy. In contrast, when amino acid (6) was added to the lines we saw strong mCherry fluorescence that was easily detectable under a dissection microscope by eye (FIG. 9 and Supplementary Movies 1-4), in a fraction of the worms (approximately 5%). Between animals in a single line we observed a large variation in both the number and identity of cells displaying mCherry fluorescence. The most likely explanations for this large variation within a line are loss of the extra-chromosomal array during developmental mitosis and/or partial silencing of the extra-chromosomal array leading, to silencing of at least one essential genetic component (synthetase or tRNA or reporter).

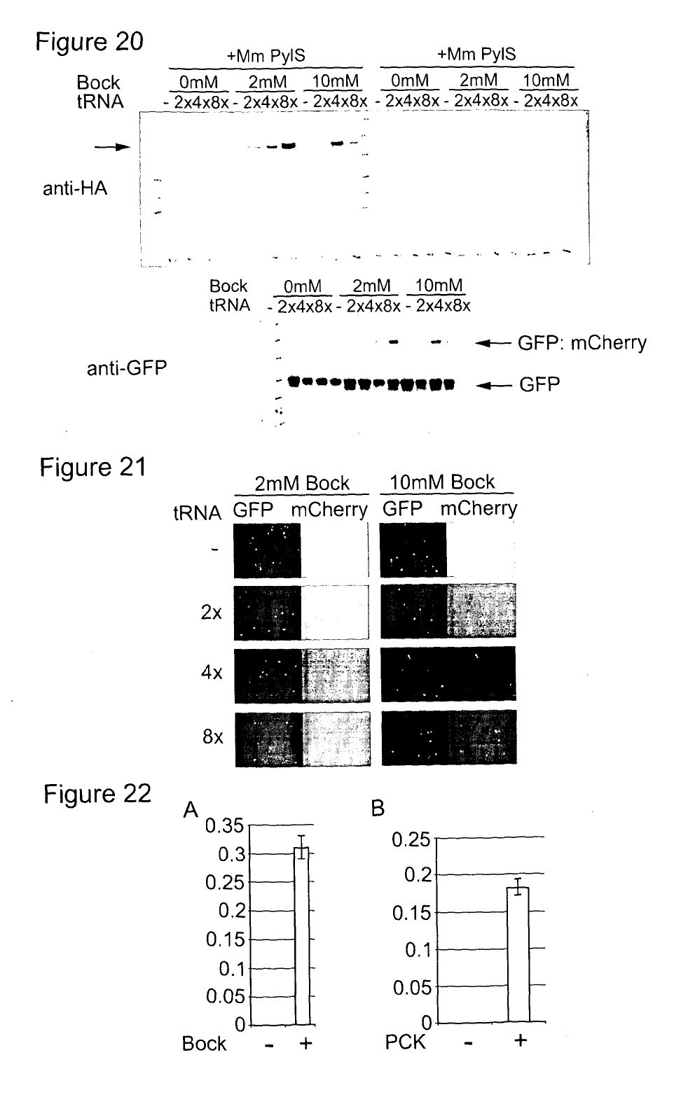

To further demonstrate that the unnatural amino acid is incorporated in response to the amber codon, leading to the production of the full length GFP-mCherry-HA-NLS, we lysed worms from each line grown in the presence and absence of (6) for western blotting. Anti-HA, and anti-GFP western blots confirmed the unnatural amino acid dependent production of GFP-mCherry-HA-NLS in worms (FIG. 9, and FIG. 11). Taken together the fluorescence imaging and western blot data demonstrate the amino acid dependent expression of mCherry, and clearly demonstrate that our transgenic strains express a functional orthogonal MmPylRS/MmtRNA_{CUA} pair that directs the incorporation of the unnatural amino acid, (6) in response to an amber stop codon in C. elegans. Preliminary experiments suggest that we can incorporate a range of unnatural amino acids in C. elegans using the approach we have reported here (data not shown).

In conclusion we have demonstrated the first genetically encoded incorporation of unnatural amino acids in a multicellular organism. Since we see mCherry expression throughout the organism our data suggest that the MmPylRS/MmtRNA_{CUA} pair can function in diverse tissues to incorporate unnatural amino acids. Since the PylRS/tRNA_{CUA} pair and its derivatives that have been evolved in E. coli can be used to direct the incorporation of a range of unnatural amino acids, extensions of the approach reported here should allow the introduction of post-translational modifications, photocaged amino acids, bioorthgonal chemical handles, and photocrosslinkers into proteins in C. elegans.

Materials and Methods

C. elegans Strains and Maintenance

Worms were grown at 20° C. on NGM agar plates according to standard protocols, unless otherwise indicated. The following alleles were used: LGI: smg-2(e2008); LGX: lin-15B(n765).

C. elegans Transformation

Transgenic lines were created by biolistic bombardment using a PDS-100/He Biolistic Particle Delivery System (Bio-Rad)¹⁻³. The bombardment mix contained 10 μg PCeN74-1::MmPylT, 10 μg Prps-0::FLAG-MmPylRS, 5 μg Prps-0::mGFP-TAG-mcherry-HA-NLS and 5 μg Prps-0::hpt. After bombardment worms were allowed to lay eggs for 36 h before adding hygromycin B to plates to a final concentration of 0.5 mg/ml. For the first 4 days bacteria were added to prevent starvation. Plates were scored for transformants after 2 weeks.

Incorporation of Unnatural Amino Acids

Worm lines were maintained on NGM plates supplemented with 1 mg/ml hygromycin B (InvivoGen). To incorporate unnatural amino acids the animals were transferred onto NGM plates without hygromycin B, supplemented with 7.5 mM amino acid (1) for 24 h to 48 h in the presence of food. Incorporation of (1) was determined by the expression of the mGFP-mCherry fusion from Prps-0::mGFP-TAG-mcherry-HA-NLS by direct fluorescence imaging or western blot of whole worm lysates.

Western Blots & Northern blots

Worms, approximately 2000, were lysed in 100 mL 4× LDS sample buffer (Invitrogen) supplemented with DTT by boiling for 15 min. After gel electrophoresis and transfer to nitrocellulose membrane the blots were probed using the following primary antibodies: anti-HA 3F10 (Roche), anti-GFP 7.1 and 13.1 (Roche), anti-FLAG M2 (Agilent). Secondary antibodies used were goat anti-rat IgG-HRP sc2065 (Santa Cruz Biotechnology) and horse anti-mouse IgG-HRP 7076 (Cell Signaling). All blocking, binding and washing steps were performed in TBS supplemented with 0.1% Tween 20 and 5% milk powder. The blots were incubated with primary antibody over night at 4° C. and with secondary antibody 1 h at room temperature. Northern blots were performed as previously described⁴, using 40 μg of total extracted RNA.

Protein Purification for Immunoprecipitations

Worms were grown on 9cm egg plates to high density¹. They were washed off the plate using M9 buffer. 0.5 ml of packed worm pellet was split equally between fresh egg plates with and without amino acid (1, 10 mM). The worms were grown on the egg plates at 20° C. for 48 h. The animals were then washed off the plates, washed once with M9 buffer, resuspended in RIPA buffer, flash frozen in liquid nitrogen and pulverized using a SPEX SamplePrep 6870 Freezer/Mill (Elvatech). After thawing, the lysate was incubated for a further 30 min at room temperature, centrifuged at 16000 g for 20 min and the supernatant incubated with RFP-trap magnetic particles (Chromotek) over night at 4° C. The particles were washed twice with 10 mM Tris pH 7.5, 300 mM NaCl and bound protein eluted by boiling in 2× LDS sample buffer (Invitrogen).

Plasmid Construction

All PCR reactions were carried out using Phusion polymerase (Finnzymes). Protein encoding constructs were assembled into pDEST R4-R3 or pDEST R4-R3_unc-54 using the Gateway system (Invitrogen). pDEST R4-R3_unc-54 contains an unc-54 3′UTR downstream of the attR3 site. Expression of all protein coding genes was driven by the rps-0 promoter (including the rps-0 ATG codon) consisting of 2.2 kb upstream of the rps-0 coding sequence, the unc-54 3′UTR was added downstream of all protein coding genes. The wild type PylRS gene from Methanosarcina mazei was amplified and an N-terminal FLAG tag introduced using primers P32/P35 and P33. An amber stop codon was introduced at the end of the mGFP coding sequence P44 and P45 and XhoI and AscI restriction enzymes. The mCherry construct was amplified using primers P158, P 159, P160 and P161 introducing a C-terminal HA tag followed by the egl-13 nuclear localization sequence⁵. The hygromycin B phosphotransferase gene (hpt), which confers resistance to hygromycin B, was amplified using primers P283 and P284.

The plasmid encoding M. mazei PylT was constructed by fusing the promoter of Ce N74.1 to PylT linked by a 2 bp sequence (AT). At the 3′ end PylT was fused to the sequence immediately downstream of C. elegans. sup-7. Primers used were P39, P40, P41, P249, P250 and P251. The PCR product was then cloned into pJet1.2 and the resulting plasmid used for transformation.

The following plasmids were constructed:

Entry Vectors:

pDONR P4-P1R
SG78 Prps-0

pDONR221
SG3 M. mazei PylRS with N-terminal FLAG tag
SG91 mGFP terminating in amber stop codon
SG107 hygromycin resistance gene

pENTR P2R-P3
SG79 mCherry-HA-elg/13NLS
SG173 unc-54 3′UTR

Constructs Used for Transformation:
SG82 (SG78+SG3+SG173+pDEST R4-R3)
SG88 (SG78+SG91+SG79+pDEST R4-R3_unc-54)
SG120 (SG78+SG107+SG173+pDEST R4-R3)
SG132 PCeN74-1_PylT sup-7 3′ (in pJet1.2)

Primer sequences

| | |
|---|---|
| 32 | TACAAGGACGACGACGACAAGGATAAAAAACCACTAAACACTCTG |
| 33 | GGGGACCACTTTGTACAAGAAAGCTGGGTTTACAGGTTGGTAGAA |
| | ATCCCGTTATAG |
| 35 | GGGGACAAGTTTGTACAAAAAAGCAGGCTATGGACTACAAGGACG |
| | ACGACGACAAG |
| 39 | CTTAAAAAACAAAAAATTCGGAAACCCCGGGAATCTAAC |
| 40 | GATTCCCGGGGTTTCCGAATTTTTTGTTTTTTAAGTAGTAATATA |
| | ATAC |
| 41 | TTGTTGCATCAGGTCTAGACAATCTG |
| 44 | CCTGGCGCGCCCTATCCTCCCTTGTAGAGCTCGTC |
| 45 | ATCTTCTTCAAGGACGACGGAAACTAC |
| 158 | TACCCATATGATGTCCCAGACTACGCTATGAGCCGTAGACGAAAA |
| | GCGA |
| 159 | AGCGTAGTCTGGGACATCATATGGGTACTTATACAATTCATCCAT |
| | GCCAC |
| 160 | GGGGACAACTTTGTATAATAAAGTTGTTAATTTTCAACTTCCTTG |
| | GCAAGCT |
| 161 | GGGGACAGCTTTCTTGTACAAAGTGGCCATGGTCTCAAAGGGTGA |
| | AGAAG |
| 249 | CAGTTTAACAAGGGCTTCAAACATCGTTC |
| 250 | CATTCGATCTACATGATCAGGTTTCCATACGGGTAGGTGATTGTA |
| | GGCTG |

(1) Berezikov, E.; Bargmann, C. I.; Plasterk, R. H. Nucleic Acids Res 2004, 32, e40.
(2) Praitis, V. Methods Mol Biol 2006, 351, 93.
(3) Praitis, V.; Casey, E.; Collar, D.; Austin, J. Genetics 2001, 157, 1217.
(4) Hancock, S. M.; Uprety, R.; Deiters, A.; Chin, J. W. J Am Chem Soc 2010, 132,14819.
(5) Lyssenko, N. N.; Hanna-Rose, W.; Schlegel, R. A. BioTechniques 2007, 43, 596.

### REFERENCES TO EXAMPLE 6

(1) Xie, J.; Schultz, P. G. Methods 2005, 36, 227.
(2) Chin, J. W.; Cropp, T. A.; Anderson, J. C.; Mukherji, M.; Zhang, Z.; Schultz, P. G. Science 2003, 301, 964.
(3) Gautier, A.; Nguyen, D. P.; Lusic, H.; An, W.; Deiters, A.; Chin, J. W. J Am Chem Soc 2010, 132, 4086.
(4) Hancock, S. M.; Uprety, R.; Deiters, A.; Chin, J. W. J Am Chem Soc 2010, 132, 14819.
(5) Mukai, T.; Kobayashi, T.; Hino, N.; Yanagisawa, T.; Sakamoto, K.; Yokoyama, S. Biochem Biophys Res Commun 2008, 371, 818.
(6) Neumann, H.; Peak-Chew, S. Y.; Chin, J. W. Nat Chem Biol 2008, 4, 232.
(7) Lammers, M.; Neumann, H.; Chin, J. W.; James, L. C. Nat Chem Biol 2010, 6, 331.
(8) Liu, C. C.; Schultz, P. G. Nat Biotechnol 2006, 24, 1436.
(9) Neumann, H.; Hancock, S. M.; Buning, R.; Routh, A.; Chapman, L.; Somers, J.; Owen-Hughes, T.; van Noort, J.; Rhodes, D.; Chin, J. W. Mol Cell 2009, 36, 153.
(10) Neumann, H.; Hazen, J. L.; Weinstein, J.; Mehl, R. A.; Chin, J. W. J Am Chem Soc 2008, 130, 4028.
(11) Nguyen, D. P.; Garcia Alai, M. M.; Kapadnis, P. B.; Neumann, H.; Chin, J. W. J Am Chem Soc 2009, 131, 14194.
(12) Nguyen, D. P.; Garcia Alai, M. M.; Virdee, S.; Chin, J. W. Chem Biol 2010, 17, 1072.
(13) Virdee, S.; Ye, Y.; Nguyen, D. P.; Komander, D.; Chin, J. W. Nat Chem Biol 2010, 6, 750.
(14) Chou, C.; Young, D. D.; Deiters, A. Chembiochem 2010, 11, 972.
(15) Gautier, A.; Deiters, A.; Chin, J. W. J Am Chem Soc 2011, 133, 2124.
(16) Lemke, E. A.; Summerer, D.; Geierstanger, B. H.; Brittain, S. M.; Schultz, P. G. Nat Chem Biol 2007, 3, 769.
(17) Carvalho, P.; Stanley, A. M.; Rapoport, T. A. Cell 2010, 143, 579.
(18) Chin, J. W.; Martin, A. B.; King, D. S.; Wang, L.; Schultz, P. G. Proc Natl Acad Sci USA 2002, 99, 11020.
(19) Hino, N.; Okazaki, Y.; Kobayashi, T.; Hayashi, A.; Sakamoto, K.; Yokoyama, S. Nat Methods 2005, 2, 201.
(20) Mori, H.; Ito, K. Proc Natl Acad Sci USA 2006, 103, 16159.
(21) Jackson, J. C.; Hammill, J. T.; Mehl, R. A. J Am Chem Soc 2007, 129, 1160.
(22) Nguyen, D. P.; Lusic, H.; Neumann, H.; Kapadnis, P. B.; Deiters, A.; Chin, J. W. J Am Chem Soc 2009, 131, 8720.
(23) Robert, V.; Bessereau, J. L. EMBO J 2007, 26, 170.
(24) C. elegans Sequencing Consortium Science 1998, 282, 2012.
(25) Sulston, J. E.; Horvitz, H. R. Dev Biol 1977, 56, 110.
(26) Kimble, J.; Hirsh, D. Dev Biol 1979, 70, 396.
(27) Kaletta, T.; Hengartner, M. O. Nat Rev Drug Discov 2006, 5, 387.
(28) Hodgkin, J. Genetics 1985, 111, 287.
(29) Li, L.; Linning, R. M.; Kondo, K.; Honda, B. M. Mol Cell Biol 1998, 18, 703.
(30) Kondo, K.; Hodgkin, J.; Waterston, R. H. Mol Cell Biol 1988, 8, 3627.
(31) Kondo, K.; Makovec, B.; Waterston, R. H.; Hodgkin, J. J Mol Biol 1990, 215, 7.
(32) Waterston, R. H. Genetics 1981, 97, 307.
(33) Hunt-Newbury, R.; Viveiros, R.; Johnsen, R.; Mah, A.; Anastas, D.; Fang, L.; Halfnight, E.; Lee, D.; Lin, J.; Lorch, A.; McKay, S.; Okada, H. M.; Pan, J.; Schulz, A. K.; Tu, D.; Wong, K.; Zhao, Z.; Alexeyenko, A.; Burglin, T.; Sonnhammer, E.; Schnabel, R.; Jones, S. J.; Marra, M. A.; Baillie, D. L.; Moerman, D. G. PLoS Blot 2007, 5, e237.
(34) Huang, L. S.; Tzou, P.; Sternberg, P. W. Mol Biol Cell 1994, 5, 395.
(35) Hodgkin, J.; Papp, A.; Pulak, R.; Ambros, V.; Anderson, P. Genetics 1989, 123,301.
(36) Longman, D.; Plasterk, R. H. A.; Johnstone, I. L.; Cáceres, J. F. Genes & Development 2007, 21, 1075.
(37) Page, M. F.; Carr, B.; Anders, K. R.; Grimson, A.; Anderson, P. Mol Cell Biol 1999, 19, 5943.
(38) Semple, J. I.; Garcia-Verdugo, R.; Lehner, B. Nat Methods 2010.
(39) Giordano-Santini, R.; Milstein, S.; Svrzikapa, N.; Tu, D.; Johnsen, R.; Baillie, D.; Vidal, M.; Dupuy, D. Nat Methods 2010, 7, 721.
(40) Gritz, L.; Davies, J. Gene 1983, 25, 179.
(41) Chin, J. W. EMBO J2011.
(42) Deng, W.; Zhu, X.; Skogerbø, G.; Zhao, Y.; Fu, Z.; Wang, Y.; He, H.; Cai, L.; Sun, H.; Liu, C.; Li, B.; Bai, B.; Wang, J.; Jia, D.; Sun, S.; He, H.; Cui, Y.; Wang, Y.; Bu, D.; Chen, R. Genome Res 2006, 16, 20.
(43) Li, T.; He, H.; Wang, Y.; Zheng, H.; Skogerbø, G.; Chen, R. BMC Mol Biol 2008, 9, 71.
(44) Praitis, V.; Casey, E.; Collar, D.; Austin, J. Genetics 2001, 157, 1217.

### Example 7

### Further Multicellular Eukaryotes

Demonstration of the invention in Drosophila systems are described.

We have tested constructs in cell culture (Drosophila S2) and subsequently used those constructs to make transgenic lines (with the constructs genomically integrated).

In the cell culture experiment we used a fluorescent reporter (GFP fused to mCherry, separated by an amber codon).

GAL4 drives expression of genes behind UAS; protein expression controlled using pMT - - - GAL4 (GAL4 driven by Metallotheine promoter - - - >expression of GAL4 is induced by addiEon of 0.5 mM Cu2+); aa - - - tRS stands for M. mazei PylRS; fusion protein which is only present in the case of incorporation of the unnatural amino acid is detected by probing with antibodies against a C-terminal HA - - - tag or by detecEng GFP (the fusion protein will be twice the size of GFP alone). Constructs are shown in FIG. 12.

Results are shown in FIG. 13. +/ - - - Mm PylS signifies presence or absence of M. mazei PylRS; 2×, 4× and 8× signify 2, 4 or 8 copies of the PylT expression casseVe

(U6 promoter+PylT+U6 3′ region) cloned into a single vector. For the anE - - - GFP blot, only the samples with PylRS are shown). The amino acid used is N_{ε} - - - (t - - - butyloxycarbonyl) - - - L - - - lysine (BocK)

In the transgenic lines the observations are consistent with incorporation of an unnatural amino acid using a Luciferase based reporter. The PylRS and reporter are cloned behind UAS promoters, it is thus possible to cross these flies with publicly available fly lines expressing GAL4 in different tissues. GAL4 induces expression of genes cloned behind UAS.

Adult flies were fed yeast supplemented with 10 mM amino acid, allowed to lay eggs, the resul/ng embryos lysed and the lysates assayed using luciferase. The expressed reporter consisted of renilla luciferase followed by firefly luciferase (the two luciferases separated by an amber stop codon. The graph shows measurements of firefly luciferase ac/vity normalised using renilla luciferase activity. Data from two independent experiments is shown. (Arbitrary fluorescence units) The animals were stably transformed with a plasmid containing Mm PylRS; luciferase reporter; 4 copies of the PylT expression cassette. PylRS and the reporter were cloned behind the UAS promoter (expression is driven by GAL4). GAL4 expression was driven by an armadillo promoter. Results are shown in FIG. 14.

Exemplary nucleic acid constructs are shown in FIGS. 15 and 16.

### Example 8

### Drosophila

Included are data showing incorporation in fly cell culture using the wild type PylRS and BocK, as well as using the PCKRS incorporating photocaged lysine (as described above and in Gautier, A., Nguyen, D. P., Lusic, H., An, W., Deiters, A., & Chin, J. W. (2010). Genetically encoded photocontrol of protein localization in mammalian cells. Journal of the American Chemical Society, 132(12), 4086-4088).

Also provided are fluorescence microscopy pictures of the cell culture experiments using GFP and mCherry as reporter.

Also provided are images for the fly embryo studies.

FIG. 18 shows N_{ε} - - - (1 - - - butyloxycarbonyl) - - - L - - - lysine (BocK) incorporation assay using Drosophila embryos. Adult flies were fed yeast supplemented with 10 mM amino acid, allowed to lay eggs, the resul/ng embryos lysed and the lysates assayed using luciferase. The expressed reporter consisted of renilla luciferase followed by firefly luciferase (the two luciferases separated by an amber stop codon. The graph shows measurements of firefly luciferase activity normalised using renilla luciferase activity. Data from two independent experiments is shown. (Arbitrary fluorescence units) The animals were stably transformed with a plasmid containing Mm PylRS; luciferase reporter; 4 copies of the PylT expression cassette. PylRS and the reporter were cloned behind the UAS promoter (expression is driven by GAL4). GAL4 expression was driven by an armadillo promoter.

FIG. 19 shows GAL4 drives expression of genes behind UAS; protein expression controlled using pMT - - - GAL4 (GAL4 driven by Metallotheine promoter - - - >expression of GAL4 is induced by addiEon of 0.5 mM Cu2+); aa - - - tRS stands for M. mazei PylRS; fusion protein which is only present in the case of incorporaEon of the unnatural amino acid is detected by probing with anEbodies against a C - - - terminal HA - - - tag or by detecEng GFP (the fusion protein will be twice the size of GFP alone)

FIG. 20 shows +/ - - - Mm PylS signifies presence or absence of M. mazei PylRS; 2×, 4× and 8× signify 2, 4 or 8 copies of the PylT expression casseVe (U6 promoter+PylT+U6 3′ region) cloned into a single vector. For the anE - - - GFP blot, only the samples with PylRS are shown). The amino acid used is N_{ε} - - - (t - - - butyloxycarbonyl) - - - L - - - lysine (BocK)

FIG. 21 shows detecEon of incorporaEon using direct fluorescence microscopy. The samples bloVed in the previous page were imaged using a fluorescence microscope.

FIG. 22 shows incorporation experiment in Drosophila cell culture (S2 cells) using Luciferase as reporter. A: using wild type PylRS to incorporate N_{ε} - - - (t - - - butyloxycarbonyl) - - - L - - - lysine (BocK) B: using PCKRS to incorporate photocaged lysine (PCK) (described in GauEer et al., 2010 GeneEcally encoded photocontrol of protein localizaEon in mammalian cells. Journal of the American Chemical Society, 132(12), 4086-4088) The same PylT cassetes as described above were used. The protein coding genes were also induced as described above for the experiment using the fluorescence reporter.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

| SEQUENCE LISTING | |
|---|---|
| MbtDNA _{CUA}^{Pyl} cassette | |
| accggtaagcttcccgataagggagcaggccagtaaaaagcattaccccgtgggaacctgatcatgtagatcgaatggactctaaatc | |
| cgttcagccgggttagattcccggggtttccgtttttttcaaaagtccctgaacttcccgctagc | |
| SNR52-MbtDNA _{CUA}^{Pyl}-SUP4 | |
| accggttctttgaaaagataatgtatgattatgctttcactcatatttatacagaaacttgatgttttctttcgagtatatacaaggtgattacatg | |
| tacgtttgaagtacaactctagattttgtagtgccctcttgggctagcggtaaaggtgcgcattttttcacaccctacaatgttctgttcaaaa | |
| gattttggtcaaacgctgtagaagtgaaagttggtgcgcatgtttcggcgttcgaaacttctccgcagtgaaagataaatgatcgggaac | |
| ctgatcatgtagatcgaatggactctaaatccgttcagccgggttagattcccggggtttccgtttttttgttttttatgtctactagt | |
| SctDN _{UCU}^{Arg}-MmtDNA _{CUA}^{Pyl} | |
| accggtatcgatgtgtgttatatgtacctctgctttgcagtataagaaatttacatttatttctgactaataacaccttggtgccccaacggtaa | |
| acaacttgtatcagttctcataagtgcggccattttatgcaatacaggctgcattatttcaccagccgtgaaaatccgaaaattgtaataatt | |
| gaaagcgtaattaggttttactataataaagtagtaaaaccttcaacaaatagtagctcgcgtggcgtaatggcaacgcgtctgacttctaa | |
| tcagaagattatgggttcgacccccatcgtgagtgctttgtttctggaaacctgatcatgtagatcgaatggactctaaatccgtttcagccg | |
| ggttagattcccggggtttccgattttttggctactcctgtagttattcttcattaatgctttgttaacgctagc | |
| SNR6ᵤₚ-MbtDNA _{CUA}^{Pyl}-SNR6_{down} | |
| accggtaaaagtatttcgtccactattttcggctactataaataaatgtttttttcgcaactatttcaacaaataagtgggaacctgatcatgta | |
| gatcgaatggactctaaatccgttcagccgggttagattcccggggtttccgtttttttttcatcgagtgaagtatcgtgacttgtacatttg | |
| aagatacccagcgtacagcagtgtatctttatcttcctgtatgatatagataactaacatctcgaatagaaaattgtctcgcgttcgaaccta | |
| agctagc | |
| Pro{TGG}FLᵤₚ-MbtDNA _{CUA}^{Pyl}-SNR6_{down} | |
| accggtcctatataaatatttctgtttttcttattaacgcaacaataattcgggaacctgatcatgtagatcgaatggactctaaatccgttcag | |
| ccgggttagattcccggggtttccgtttttttttatcatcgagtgaagtatcgtgacttgtacatttgaagatacccagcgtacagcagtgtat | |
| ctttatcttcctgtatgatatagataactaacatctcgaatagaaaattgtctcgcgttcgaacctaagctagc | |
| Ile{TAT}LR1ᵤₚ-MbtDNA _{CUA}^{Pyl}-SNR6_{down} | |
| accggtcaagccggaactcaaaagggtaatttcgtgaaaaacaatcatctacggtataaataacaatttaatttacgtctctttcgaaaatg | |
| ggaacctgatcatgtagatcgaatggactctaaatccgttcagccgggttagattcccggggtttccgtttttttttcatcgagtgaagtat | |
| cgtgacttgtacatttgaagatacccagcgtacagcagtgtctttatcttcctgtatgatatagataactaacatctcgaatagaaaattgt | |
| ctcgcgttcgaacctaagctagc | |
| Asp{GTC}KRᵤₚ-Mbt DNA _{CUA}^{Pyl}-SNR6_{down} | |
| accggtgaaatataaatatttaaaactaagagaaaaaatccaacaaataacgtgggaacctgatcatgtagatcgaatggactctaaatc | |
| cgttcagccgggttagattcccggggtttccgtttttttttatcatcgagtgaagtatcgtgacttgtacatttgaagatacccagcgtacagc | |
| agtgtatctttatcttcctgtatgatatagataactaacatctcgaatagaaaattgtctcgcgttcgaacctaagtagc | |
| Primers | |
| P59 | cccggggtttccgccatttttttcaaaagtc |
| P60 | gggacttttgaaaaaaatggcggaaaccccg |
| P82 | cgttcagccgggttcgattcccgggg |
| P83 | gaaaccccgggaatcgaacccggctg |
| P84 | cccgtgggaacctgctcaggtagagcgaatggactc |
| P85 | gatttagagtccattcgctctacctgagcaggttccc |
| P88 | gggaacctgatcatgtagatcgaatggactctaaatccgttcagccgggttagattcccggggtttccg |
| P164 | accggtatcgatgtgtgttatatgtacctctgctttgcagtataagaaatttacatttatttc |
| P165 | gttgtttaccgttggggcaccaaggtgttattagtcagaaataaatgtaaatttcttatactg |
| P166 | gccccaacggtaaacaacttgtatcagttctcataagtgcggccattttatgcaatacaggc |
| P167 | caattactacaattttcggattttcacggctggtgaaataatgcagcctgtattgcataaaatgg |
| P168 | gaaaatccgaaaattgtagtaattgaaagcgtaattaggttttactataataaagtagtaaaacc |
| P169 | cgttgccattacgccacgcgagctactatttgttgaaggttttactactttattatagtaaaac |
| P170 | gtggcgtaatggcaacgcgtctgacttctaatcagaagattatgggttcgacccccatcgtgag |
| P171 | gatttagagtccattcgatctacatgatcaggttcccagaaacaaagcactcacgatgggggtcg |
| P172 | gatcgaatggactctaaatccgttcagccgggttagattcccggggtttccgatttttttggc |
| P173 | gctagcgttaacaaagcattaatgaagaataactacaggagtagccaaaaaaatcggaaaccc |
| P183 | ggtggtaccggtaaaagtatttcgtccactattttcggctactataaataaatgtttttttcgcaac |
| P184 | gatctacatgatcaggttcccacttatttgttgaaatagttgcgaaaaaaacatttatttatag |
| P186 | cggaaccccgggaatctaac |
| P187 | gattcccggggtttccgtttttttttcatcgagtgaagtatcgtg |
| P188 | ggtggtgctagcttaggttcgaacgcgagacaattttc |
| P189 | ggtggtaccggtcaagccggaactcaaaagggtaatttcgtgaaaaacaatcatctacggtataaataac |
| P190 | gatctacatgatcaggttcccattttcgaaagagacgtaaattaaattgttatttataccgtagatgattg |
| P191 | ggtggtaccggtcctatataaatatttctgtttttcttattaacgcaacaataattcgggaacctgatcatgtagatc |
| P192 | ggtggtaccggtgaaatataaatatttaaaactaagagaaaaaatccaacaaataacgtgggaacctgatcatgtagatc |
| P202 | gagtgctttgtttctggaaacctgatcatgtag |
| P203 | catgatcaggtttccagaaacaaagcactcacg |
| P217 | ccggtccggatacatatgctcctttcg |
| P218 | ctagcgaaaggagcatatgtatccgga |
| P238 | gtgtttaagaccaatggtggctccaactatttttaattacgccagaaagttgg |
| P239 | ctatccaactttctggcgtaattaaaaatagttggagccaccattggtcttaaac |
| P240 | ctatggttaacttctttcaaatgggttctggttg |
| P241 | gtacaaccagaacccatttgaaagaagttaacc |
| P242 | gtttaagaccaatgttggctccaactattttgaattacgccag |
| P243 | ctttctggcgtaattcaaaatagttggagccaacattggtct |
| P244 | gattccagctgaatacgttgaaagattcggtattaacaacg |
| P245 | gtatcgttgttaataccgaatctttcaacgtattcagctgg |
| P246 | gtttaagaccaatgttgtctccaactttgtgtaattacatgagaaagttgg |
| P247 | ctatccaactttctcatgtaattacacaaagttggagacaacattggtc |
| P287 | cttgtgtttaagaccaatgatggctccaactatt |
| P288 | gttggagccatcattggtcttaaacacaagttc |

1. A nucleic acid comprising a nucleotide sequence encoding a tRNA orthogonal to a eukaryotic cell, said nucleotide sequence operably linked to a promoter capable of directing transcription by eukaryotic RNA polymerase III.

2. A nucleic acid according to claim 1 wherein said orthogonal tRNA is tRNA^{Pyl}.

3. A nucleic acid according to claim 1 wherein said promoter is, or is derived from, the eukaryotic U6 promoter.

4. A nucleic acid according to claim 1 wherein said eukaryotic cell is a yeast cell and wherein the tRNA^{Pyl} comprises sequence at positions 3 and 70 which do not form a 3-70 base pair.

5. A nucleic acid according to claim 4 wherein the tRNA^{Pyl} comprises adenosine at position 3.

6. A nucleic acid according to claim 4 wherein the yeast is Saccharomyces cerevisiae.

7. A nucleic acid according to claim 1 wherein the promoter comprises A and B box consensus sequences.

8. A nucleic acid according to claim 1 wherein the promoter comprises the yeast sequence encoding tRNA^{Arg}_{UCU}.

9. A nucleic acid according to claim 1 wherein the tRNA^{Pyl} is tRNA^{Pyl}_{CUA}.

10. A nucleic acid according to claim 9 wherein the sequence encoding tRNA^{Pyl}_{CUA} comprises the M. mazei tRNA^{Pyl}_{CUA} sequence.

11. A nucleic acid according to claim 9 wherein the sequence encoding tRNA^{Pyl}_{CUA} comprises the M. barkeri tRNA^{Pyl}_{CUA} sequence having a G3A substitution.

12. An expression system comprising a nucleic acid according to claim 1; said system further comprising a nucleotide sequence encoding a PylRS capable of aminoacylating the tRNA^{Pyl}.

13. An expression system according to claim 12 wherein the PylRS comprises M. barkeri PylRS or AcKRS or TfaKRS or PcKRS.

14. A eukaryotic cell comprising a nucleic acid according to claim 1.

15. The eukaryotic cell according to claim 14, wherein the eukaryotic cell is a yeast cell and wherein said yeast is S. cerevisiae.

16. The eukaryotic cell according to claim 14, wherein the eukaryotic cell is a C. elegans cell.

17. The eukaryotic cell according to claim 14, wherein the eukaryotic cell is a fly cell such as a Drosophila cell.

18. The eukaryotic cell according to claim 14, wherein the eukaryotic cell is a mammalian cell such as a mouse or human cell.

19. (canceled)

20. A method for incorporating an unnatural amino acid into a protein in a eukaryotic cell such as a yeast cell comprising the following steps:
i) introducing an expression system according to claim 12 into said cell;
ii) introducing a nucleic acid encoding the protein into said cell, said nucleic acid comprising an orthogonal codon recognised by the tRNA^{Pyl} of the expression system at the position for incorporation of the unnatural amino acid; and
iii) incubating the cell in the presence of unnatural amino acid to be incorporated.

21. The method according to claim 20, wherein the unnatural amino acid to be incorporated is an alkyne-containing amino acid or a post-translationally modified amino acid or an amino acid containing bio-orthogonal chemical handles or a photo-caged amino acid or a photo-crosslinking amino acid.
